(19) United States
(12) Patent Application Publication
(10) Pub. No.: US 2022/0316014 A1
(43) Pub. Date: Oct. 6, 2022

(54) METHODS FOR DIAGNOSING THE EFFECTIVENESS OF ANTI-TUMOR TREATMENT

(71) Applicant: Intellexon GmbH, Pöcking (DE)

(72) Inventors: Wolfgang WÜRFEL, Odelzhausen (DE); Ralph Markus WIRTZ, Köln (DE); Christoph WINTERHALTER, Pöcking (DE); Franziska WÜRFEL, Odelzhausen (DE)

(21) Appl. No.: 17/624,829

(22) PCT Filed: Jul. 6, 2020
(86) PCT No.: PCT/EP2020/068990

§ 371 Date: Jan. 4, 2022

(30) Foreign Application Priority Data
Jul. 5, 2019 (EP) 19184681.5

(51) Int. Cl. C12Q 1/6886; C07K 16/28; C12N 9/22; C12N 15/11

(52) U.S. Cl. CPC C12Q 1/6886; C07K 16/2818; C12N 9/22; C12N 15/111; C12Q 2600/106; C12Q 2600/158; C12N 2310/20

(57) ABSTRACT

The present invention relates to a method for predicting whether a subject having a tumor responds to a tumor therapy selected from (i) an immunotherapy, (ii) a chemotherapy, (iii) an anti-hormonal therapy, and (iv) an anti-tyrosin kinase therapy, wherein the method comprises (A) determining the level(s) of at least one nucleic acid molecule and/or at least one protein or peptide in a sample obtained from said subject, wherein the at least one nucleic acid molecule is selected from nucleic acid molecules (a) encoding a polypeptide comprising or consisting of the amino acid sequence of any one of SEQ ID NOs 1 to 6, (b) consisting of the nucleotide sequence of any one of SEQ ID NOs 7 to 12, (c) encoding a polypeptide which is at least 85% identical, preferably at least 90% identical, and most preferred at least 95% identical to the amino acid sequence of (a), (d) consisting of a nucleotide sequence which is at least 95% identical, preferably at least 96% identical, and most preferred at least 98% identical to the nucleotide sequence of (b), (e) consisting of a nucleotide sequence which is degenerate with respect to the nucleic acid molecule of (d), (f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and (g) corresponding to the nucleic acid molecule of any one of (a) to (f), wherein T is replaced by U, and wherein the at least one protein or peptide is selected from proteins or peptides being encoded by the nucleic acid molecule of any one of (a) to (g); and (B) comparing the level(s) of (A) with the level(s) of the at least one nucleic acid molecule and/or the at least one protein or peptide in a sample obtained from one or more subjects that responded to one or more of the therapies of (i) to (ii) or a corresponding pre-determined standard, wherein increased level(s) of (A) as compared to the level(s) or pre-determined standard of (B) indicate(s) that the subject will not respond to the tumor therapy and substantially the same or decreased level(s) of (A) as compared to the level(s) of (B) indicate(s) that the subject will respond to the tumor therapy; or (B′) comparing the level(s) of (A) with the level(s) of the at least one nucleic acid molecule and/or the at least one protein or peptide in a sample obtained from one or more subjects that did not respond to one or more of the therapies of (i) to (iii) or a corresponding pre-determined standard, wherein decreased level(s) of (A) as compared to the level(s) or pre-determined standard of (B′) indicate(s) that the subject will respond to the tumor therapy and substantially the same or increased level(s) of (A) as compared to the level(s) of (B′) indicate(s) that the subject will not respond to the tumor therapy.

### RELATED PATENT APPLICATIONS

This patent application is a National Phase entry of, and claims priority to International Patent Application No. PCT/EP2020/068990 filed Jul. 6, 2020, entitled METHODS FOR DIAGNOSING THE EFFECTIVENESS OF ANTI-TUMOR TREATMENT, which claims priority to European Patent Application No. 19184681.5 filed Jul. 5, 2019. The entire content of the foregoing patent applications is incorporated herein by reference, including all text, tables and drawings.

### SEQUENCE LISTING

The instant application contains a Sequence Listing which has been submitted electronically in ASCII format and is hereby incorporated by reference in its entirety. Said ASCII copy, created on Dec. 9, 2021, is named “009848-0565249_sequence_listing” and is 41.7 KB in size.

The present invention relates to a method for predicting whether a subject having a tumor responds to a tumor therapy selected from (i) an immunotherapy, (ii) a chemotherapy, (iii) an anti-hormonal therapy, and (iv) an anti-tyrosin kinase therapy, wherein the method comprises (A) determining the level(s) of at least one nucleic acid molecule and/or at least one protein or peptide in a sample obtained from said subject, wherein the at least one nucleic acid molecule is selected from nucleic acid molecules (a) encoding a polypeptide comprising or consisting of the amino acid sequence of any one of SEQ ID NOs 1 to 6, (b) consisting of the nucleotide sequence of any one of SEQ ID NOs 7 to 12, (c) encoding a polypeptide which is at least 85% identical, preferably at least 90% identical, and most preferred at least 95% identical to the amino acid sequence of (a), (d) consisting of a nucleotide sequence which is at least 95% identical, preferably at least 96% identical, and most preferred at least 98% identical to the nucleotide sequence of (b), (e) consisting of a nucleotide sequence which is degenerate with respect to the nucleic acid molecule of (d), (f) consisting of a fragment of the nucleic acid molecule or any one of (a) to (e), said fragment comprising at least 150 nucleotides, preferably at least 300 nucleotides, more preferably at least 450 nucleotides, and most preferably at least 600 nucleotides, and (g) corresponding to the nucleic acid molecule of any one of (a) to (f), wherein T is replaced by U, and wherein the at least one protein or peptide is selected from proteins or peptides being encoded by the nucleic acid molecule of any one of (a) to (g); and (B) comparing the level(s) of (A) with the level(s) of the at least one nucleic acid molecule and/or the at least one protein or peptide in a sample obtained from one or more subjects that responded to one or more of the therapies of (i) to (iii) or a corresponding pre-determined standard, wherein increased level(s) of (A) as compared to the level(s) or pre-determined standard of (B) indicate(s) that the subject will not respond to the tumor therapy and substantially the same or decreased level(s) of (A) as compared to the level(s) of (B) Indicate(s) that the subject will respond to the tumor therapy; or (B′) comparing the level(s) of (A) with the level(s) of the at least one nucleic acid molecule and/or the at least one protein or peptide in a sample obtained from one or more subjects that did not respond to one or more of the therapies of (i) to (iii) or a corresponding pre-determined standard, wherein decreased level(s) of (A) as compared to the level(s) or pre-determined standard of (B) indicate(s) that the subject will respond to the tumor therapy and substantially the same or increased level(s) of (A) as compared to the level(s) of (B) indicate(s) that the subject will not respond to the tumor therapy.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The human leukocyte antigen (HLA) system or complex is a gene complex encoding the major histocompatibilty complex (MHC) proteins in humans. These cell-surface proteins are responsible for the regulation of the immune system in humans. The HLA gene complex resides on a 3 Mbp stretch within chromosome 6p21. Genes in this complex are categorized into three basic groups: class I, class II, and class III.

Humans have three main MHC class I genes, known as HLA-A, HLA-B, and HLA-C. The proteins produced from these genes are present on the surface of almost all cells. On the cell surface, these proteins are bound to protein fragments (peptides) that have been exported from the inside of the cell. MHC class I proteins display these peptides to the immune system. If the immune system recognizes the peptides as foreign (such as viral or bacterial peptides), it responds by triggering the infected cell to self-destruction.

There are six main MHC class II genes in humans: HLA-DPA1, HLA-DPB1, HLA-DQA1, HLA-DQB1, HLA-DRA, and HLA-DRB1. MHC class II genes provide instructions for making proteins that are present almost exclusively on the surface of certain immune system cells. Like MHC class I proteins, these proteins display peptides to the immune system.

The proteins produced from MHC class III genes have somewhat different functions; they are involved in inflammation and other immune system activities. The functions of some MHC genes are unknown.

HLA genes have many possible variations, allowing each person's immune system to react to a wide range of foreign invaders. Some HLA genes have hundreds of identified versions (alleles), each of which is given a particular number (such as HLA-B27). Closely related alleles are categorized together; for example, at least 40 very similar alleles are subtypes of HLA-B27. These subtypes are designated as HLA-B*2701 to HLA-B*2743.

More than 100 diseases have been associated with different alleles of HLA genes. For example, the HLA-B27 allele increases the risk of developing an inflammatory joint disease called ankylosing spondylitis. Many other disorders involving abnormal immune function and some forms of cancer have also been associated with specific HLA alleles. However, it is often unclear what role HLA genes play in the risk of developing these diseases.

Next to the three main MHC class I genes the non-classical MHC class I molecules HLA-E, HLA-F HLA-G are encoded by the HLA class I region. The overexpression of HLA-G, -E, and -F is a common finding across a variety of malignancies (Kochan et al., Oncoimmunology. 2013 Nov. 1; 2(11): e26491.). HLA-G and HLA-E were reported as being cancer biomarkers and also as being positively correlated with poor clinical outcome of cancer.

The HLA class I region was furthermore reported to include class I pseudogenes (Hughes, Mol Biol Evol. 1995 March; 12(2):247-58) as well as gene fragments. For instance. HLA-H, J and L are classified as class I pseudogenes and HLA-N, S and X are classified as gene fragments. In particular, it was reported by Messer et al., J Immunol. 1992 Jun. 15; 148(12):4043-53 that HLA-J is a pseudogene, due to deleterious mutations that produce a translation termination either in exon 2 or exon 4. Hence, human leukocyte antigen (HLA) genes have a long research history as important targets in biomedical science, diagnosis and treatment.

Moreover, cancer is the second leading cause of death globally, and is responsible for an estimated 9.6 million deaths in 2018. Globally, about 1 in 8 deaths is due to cancer. The incidence of cancer is currently even increasing, inter alia, due to people becoming older and older. Cancer mortality can be reduced if cases are detected and treated early. In the absence of early diagnosis, patients are diagnosed at late stages when curative treatment may no longer be an option. However, even if the cancer is diagnosed at an early stage the heterogeneity of tumors still often makes the finding of an efficient treatment for a particular patient difficult. This is because the bulk tumour might include a diverse collection of cells harbouring distinct molecular signatures with differential levels of sensitivity to treatment. This heterogeneity might result in a non-uniform distribution of genetically distinct tumour-cell subpopulations across and within disease sites (spatial heterogeneity) or temporal variations in the molecular makeup of cancer cells (temporal heterogeneity). Heterogeneity provides the fuel for resistance of the tumor to certain treatment options. Therefore, there is an urgent need for predicting in advance whether a subject having a tumor responds to a particular tumor therapy or not. Also there is an urgent need for new tumor therapies. These needs are addressed by the present invention.

Accordingly, the present invention relates in a first aspect to a method for predicting whether a subject having a tumor responds to a tumor therapy selected from (i) an immunotherapy, (ii) a chemotherapy, (iii) an anti-hormonal therapy, and (iv) an anti-tyrosin kinase therapy, wherein the method comprises (A) determining the level(s) of at least one nucleic acid molecule and/or at least one protein or peptide in a sample obtained from said subject, wherein the at least one nucleic acid molecule is selected from nucleic acid molecules (a) encoding a polypeptide comprising or consisting of the amino acid sequence of any one of SEQ ID NOs 1 to 6, (b) consisting of the nucleotide sequence of any one of SEQ ID NOs 7 to 12, (c) encoding a polypeptide which is at least 85% identical, preferably at least 90% identical, and most preferred at least 95% identical to the amino acid sequence of (a), (d) consisting of a nucleotide sequence which is at least 95% identical, preferably at least 96% identical, and most preferred at least 98% identical to the nucleotide sequence of (b), (e) consisting of a nucleotide sequence which is degenerate with respect to the nucleic acid molecule of (d), (f) consisting of a fragment of the nucleic acid molecule of any one of (a) to (e), said fragment comprising at least 150 nucleotides, preferably at least 250 nucleotides, more preferably at least 300 nucleotides, even more preferably at least 450 nucleotides, and most preferably at least 800 nucleotides, and (g) corresponding to the nucleic acid molecule of any one of (a) to (f), wherein T is replaced by U. and wherein the at least one protein or peptide is selected from proteins or peptides being encoded by the nucleic acid molecule of any one of (a) to (g); and (B) comparing the level(s) of (A) with the level(s) of the at least one nucleic acid molecule and/or the at least one protein or peptide in a sample obtained from one or more subjects that responded to one or more of the therapies of (i) to (iii) or a corresponding pre-determined standard, wherein increased level(s) of (A) as compared to the level(s) or pre-determined standard of (B) indicate(s) that the subject will not respond to the tumor therapy and substantially the same or decreased level(s) of (A) as compared to the level(s) of (B) indicate(s) that the subject will respond to the tumor therapy; or (B) comparing the level(s) of (A) with the level(s) of the at least one nucleic acid molecule and/or the at least one protein or peptide in a sample obtained from one or more subjects that did not respond to one or more of the therapies of (i) to (ii) or a corresponding pre-determined standard, wherein decreased level(s) of (A) as compared to the level(s) or pre-determined standard of (B′) indicate(s) that the subject will respond to the tumor therapy and substantially the same or increased level(s) of (A) as compared to the level(s) of (B′) indicate(s) that the subject will not respond to the tumor therapy.

The term “subject” in accordance with the invention refers to a mammal, preferably a domestic animal or a pet animal such as horse, cattle, pig, sheep, goat, dog or cat, and most preferably a human.

A tumor is an abnormal benign or malignant new growth of tissue that possesses no physiological function and arises from uncontrolled usually rapid cellular proliferation. The tumor is preferably cancer. Cancer is an abnormal malignant new growth of tissue that possesses no physiological function and arises from uncontrolled usually rapid cellular proliferation. The cancer is preferably selected from the group consisting of breast cancer, ovarian cancer, endometrial cancer, vaginal cancer, vulva cancer, bladder cancer, salivary gland cancer, endometrium cancer, pancreatic cancer, thyroid cancer, kidney cancer, lung cancer, cancer concerning the upper gastrointestinal tract, colon cancer, colorectal cancer, prostate cancer, squamous-cell carcinoma of the head and neck, cervical cancer, glioblastomas, malignant ascites, lymphomas and leukemias. Preferred cancers will be defined herein below.

The tumor or cancer is preferably a solid tumor or cancer. A solid tumor or cancer is an abnormal mass of tissue that usually does not contain cysts or liquid areas by contrast to a non-solid tumor (e.g. leukemia).

While a tumor therapy may in general also be, for example, a surgery, the tumor therapy herein is selected from (i) an immunotherapy, (ii) a chemotherapy, (iii) an anti-hormonal therapy, and (iv) an anti-tyrosin kinase therapy. Among these tumor therapies an immunotherapy is preferred.

An immunotherapy is the treatment of a disease by activating or suppressing the immune system. In accordance with the present invention the immunotherapy is to treat a tumor and hence the immunotherapy is a tumor immunotherapy, preferably a cancer immunotherapy. Tumor immunotherapy is in general terms the artificial stimulation of the immune system to treat the tumor, improving on the system's natural ability to fight the tumor. Immunotherapy can be categorized as active, passive or hybrid (active and passive). Active immunotherapy directs the immune system to attack tumor cells by targeting tumor antigens. Passive immunotherapies enhance existing anti-tumor responses and include, for example, the use of monoclonal antibodies, lymphocytes and cytokines.

The immunotherapy preferably comprises the application of an immune checkpoint inhibitor and the immunotherapy is accordingly preferably an immune checkpoint inhibitor therapy. Immune checkpoint inhibitors (also known as simply checkpoint inhibitors) are drugs that help the immune system to respond more strongly to a tumor. These drugs work, for example, by releasing “brakes” that keep T cells (a type of white blood cell and part of the immune system) from killing tumor cells. Such drugs do not target the tumor directly. Instead, they interfere with the ability of tumor cells to avoid an immune system attack against the tumor cells.

Immune checkpoints therefore affect immune system function. Immune checkpoints can be stimulatory or inhibitory. Tumors can use these checkpoints to protect themselves from immune system attacks. Stimulatory checkpoint molecules are, for example, members of the tumor necrosis factor (TNF) receptor superfamily (CD27, CD40, OX40, GITR and CD137) and molecules belonging to the B7-CD28 superfamily (CD28 itself and ICOS). Inhibitory checkpoint molecules are, for example, CD20, CD28, CD80, CD88. CD137, IDO1, LAG3, TIM3, TIM-4, TIGIT, BTLA, OX40, VISTA, B7-H7, CD27, GITR, CTLA4 and PD-1 and PD-L1. Currently approved checkpoint therapies mostly block inhibitory checkpoint receptors. Blockade of negative feedback signaling to immune cells thus results in an enhanced immune response against the tumor. Non-limiting but preferred examples of immune checkpoints and inhibitors thereof will be provided and discussed herein below. Inhibition and/or activation of checkpoints might be achieved by affecting singular targets or combinations thereof. By way of illustration but not of limitation this might be a combination of anti-CTLA4 and/or PD-1 and/or PD-L1. Moreover the efficacy of checkpoint inhibitors might be improved by additional treatment using chemotherapeutic, and/or hormonal and/or receptor tyrosine kinase inhibitors and/or DNA damage repair inhibitors.

A chemotherapy is a cancer therapy that uses drugs called cytostatics, which aim to stop tumor cells from continuing to divide uncontrollably. The cytostatics are usually administered via infusion into a vein, but some they can also be taken as tablets. Chemotherapy may be given with a curative intent (which almost always involves combinations of drugs), or it may aim to prolong life or to reduce symptoms (palliative chemotherapy). Cytostatics may act, for example, via the inhibition of nucleic acid synthesis, damage of nucleic acid, or alteration of microtubular protein (spindle poisons), or cell membrane damage. Chemotherapy is often combined with radiotherapy—this is then called radiochemotherapy. The chemotherapy as referred to herein may be an adjuvant chemotherapy or a neoadjuvant chemotherapy, and is preferably a neoadjuvant chemotherapy. In neoadjuvant (also called preoperative or primary) chemotherapy, drug treatment takes place before surgical extraction of a tumor. This is in contrast with adjuvant chemotherapy, which is drug treatment after surgery. The efficacy of chemotherapeutic agents might release tumor antigens by cell destruction which are then presented to the immune system, which might ultimatively lead to increased recognition by the immune system thereby increasing effectiveness of immunotherapeutic agents such as immune check point inhibitors or activators.

An anti-hormonal therapy is a treatment that blocks the production or action of a hormone. An anti-hormonal therapy is useful in tumor treatment because certain hormones are able to stimulate the growth of some types of tumors. For example, endocrine therapy of mammary and prostate cancer has been long established. The therapies available to block sex-hormone-receptor-mediated tumor growth are based on two principles: (i) ligand depletion, which can be achieved surgically, by use of luteinizing hormone-releasing hormone analogues or inhibitors of enzymes involved in steroid biosynthesis or by interfering with the feedback mechanisms of sex hormone synthesis at the pituitary/hypothalamic level; and (ii) blockade of sex hormone receptor function by use of antihormones. For example, Tamoxifen is used for the treatment of breast cancer and blocks estrogen receptors on breast cancer cells. In addition, anti-hormonal and/or hormonal treatment also affect the immune system and the presentation of antigens, which might be of importance for immune modulatory treatment strategies. The interaction of hormone activities/dependencies and HLA factors have been investigated as part of the invention.

An anti-tyrosin kinase therapy uses a tyrosine kinase inhibitor (TKI) being a pharmaceutical drug that inhibits tyrosine kinases. Tyrosine kinases are enzymes responsible for the activation of many proteins by signal transduction cascades. The proteins are activated by adding a phosphate group to the protein (phosphorylation), a step that TKIs inhibit. TKIs are used as anticancer drugs. TKIs operate by four different mechanisms: they can compete with adenosine triphosphate (ATP), the phosphorylating entity, the substrate or both or can act in an allosteric fashion, namely bind to a site outside the active site, affecting its activity by a conformational change. The interaction of receptor tyrosine kinases and HLA factors have been investigated as part of the invention.

The nucleic acid sequences of SEQ ID NOs 7 to 12 are the genes of the human HLA genes membrane-bound HLA-G, HLA-L, soluble HLA-G, HLA-H, HLA-J, and HLA-L, respectively. In addition, the membrane bound isoforms can be released by proteolytic activity, thereby increasing the soluble fraction of HLA-G and HLA-L. It is preferred that the nucleic acid molecule according to the invention is genomic DNA or mRNA. In the case of mRNA, the nucleic acid molecule may in addition comprise a poly-A tail.

As surprisingly found in accordance with the invention and shown in the examples herein below, HLA-G is expressed as a full-length transcript and a splice form only comprising exons 1 to 5 of HLA-G. While full-length HLA-G comprises a transmembrane domain and is thus membrane-bound, soluble HLA-G lacks this transmembrane domain. It is furthermore shown in the examples that a high level of the expression of the mRNA encoding full-length HLA-G (i.e., for example, Indicated by high-levels of expression measured for exons 5 and 8 or only for exon 8) as well as a high expression of the mRNA encoding the soluble form (i.e., for example, Indicated by high-levels of expression measured for exons 5 and low level of exon 8, or only high level of exon 5) is associated with a tumor patient not responding to a tumor therapy as defined herein above. As depicted above, the membrane bound HLA isoforms can also be released by post-translational proteolytic cleavage to result in the release of soluble HLA fragments.

Also the gene encoding HLA-L comprises a sequence encoding a transmembrane domain. It is therefore believed that also HLA-L can be found in tumors in a full-length membrane-bound form (SEQ ID NO: 2) as well as a soluble form (SEQ ID NO: 8). Full-length HLA-L might also be released by post-translational proteolytic cleavage to result in the release of soluble HLA fragments.

On the other hand, the genes encoding HLA-H and HLA-J (SEQ ID NOs 11 and 12) do not comprise an open reading frame encoding a transmembrane domain. It is shown in the examples herein below that HLA-H and HLA-J are soluble. The examples herein below also show that a high expression of the mRNA encoding such soluble HLAs is associated with a tumor patient not responding to a tumor therapy as defined herein above.

SEQ ID NOs 1 to 6 are the amino acid sequences of human HLA genes HLA-G, HLA-L, soluble HLA-G, HLA-H, HLA-J and HLA-L protein, respectively.

The term “nucleic acid sequence” or “nucleic acid molecule” in accordance with the present invention includes DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases, such as mRNA. Included are also single- and double-stranded hybrids molecules. i.e., DNA-DNA, DNA-RNA and RNA-RNA. The nucleic acid molecule may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation or a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule may additionally comprise promoters, enhancers, response elements, signal sequences, polyadenylation sequences, introns, 5′- and 3′-non-coding regions, and the like.

The term “protein” as used herein interchangeably with the term “polypeptide” describes linear molecular chains of amino acids, including single chain proteins or their fragments, containing at least 50 amino acids. The term “peptide” as used herein describes a group of molecules consisting of up to 49 amino acids, whereas the term “polypeptide” (also referred to as “protein”) as used herein describes a group of molecules consisting of at least 50 amino acids. The term “peptide” as used herein describes a group of molecules consisting with increased preference of at least 15 amino acids, at least 20 amino acids at least 25 amino acids, and at least 40 amino acids. The group of peptides and polypeptides are referred to together by using the term “(poly)peptide”. (Poly)peptides may further form oligomers consisting of at least two identical or different molecules. The corresponding higher order structures of such multimers are, correspondingly, termed homo- or heterodimers, homo- or heterotrimers etc. For example, the HLA proteins comprise cysteins and thus potential dimerization sites. Furthermore, peptidomimetics of such proteins/(poly)peptides where amino acid(s) and/or peptide bond(s) have been replaced by functional analogues are also encompassed by the invention. Such functional analogues include all known amino acids other than the 20 gene-encoded amino acids, such as selenocysteine. The terms “(poly)peptide” and “protein” also refer to naturally modified (poly)peptides and proteins where the modification is effected e.g. by glycosylation, acetylation, phosphorylation and similar modifications which are well known in the art.

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences. In other terms, using an alignment for two or more sequences or subsequences the percentage of amino acid residues or nucleotides that are the same (e.g. 80%, 85%, 90% or 95% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned.

Nucleotide and amino acid sequence analysis and alignment in connection with the present invention are preferably carried out using the NCBI BLAST algorithm (Stephen F. Altschul, Thomas L. Madden, Alejandro A. Schäffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid sequences.

As defined herein, sequence identities or at least 85% identity, preferably at least 90% identity, and most preferred at least 95% identity are envisaged by the invention. However, also envisaged by the invention are with increasing preference sequence identities of at least 97.5%, at least 98.5%, at least 99%, at least 99.5%, at least 99.8%, and 100% identity.

The sample may be a body fluid of the subject or a tissue sample from an organ of the subject. Non-limiting examples of body fluids are whole blood, blood plasma, blood serum, urine, peritoneal fluid, and pleural fluid, liquor cerebrospinalis, tear fluid, or cells therefrom in solution. Non-limiting examples of tissue are colon, liver, breast, ovary, and testis. Tissue samples may be taken by aspiration or punctuation, excision or by any other surgical method leading to biopsy or resected cellular material. The sample may be a processed sample, e.g. a sample which has been frozen, fixed, embedded or the like. A preferred type of sample is a formaline fixed paraffin embedded (FFPE) sample. Preparation of FFPE samples are standard medical practice and these samples can be conserved for long periods of time.

Methods for obtaining the levels of the nucleic acid molecule or the protein or peptide in the context of the method the invention are established in the art.

For instance, levels of the nucleic acid molecule may be obtained by real time quantitative PCR (RT-qPCR), electrophoretic techniques or a DNA Microarray (Roth (2002). Curr. Issues Mol. Biol., 4: 93-100), wherein a RT-qPCR is preferred. In these methods the expression level may be normalized against the (mean) expression level of one or more reference genes in the sample. The term “reference gene”, as used herein, is meant to refer to a gene which has a relatively invariable level of expression on the RNA transcript/mRNA level in the system which is being examined, i.e. the tumor. Such a gene may be referred to as a housekeeping gene. Non-limiting examples of reference genes are CALM2, B2M, RPL37A, GUSB, HPRT1 and GAPDH, preferably CALM2 and/or B2M. Other suitable reference genes are known to a person skilled in the art.

RT-qPCR is carried out in a thermal cycler with the capacity to illuminate each sample with a beam of light of at least one specified wavelength and detect the fluorescence emitted by the excited fluorophore. The thermal cycler is also able to rapidly heat and chill samples, thereby taking advantage of the physicochemical properties of the nucleic acids and DNA polymerase. The two common methods for the detection of PCR products in real-time qPCR are: (1) non-specific fluorescent dyes that intercalate with any double-stranded DNA, and (2) sequence-specific DNA probes consisting of oligonucleotides that are labelled with a fluorescent reporter which permits detection only after hybridization of the probe with its complementary sequence (e.g. a TaqMan probe). The probes are generally fluorescently labeled probes. Preferably, a fluorescently labeled probe consists of an oligonucleotide labeled with both a fluorescent reporter dye and a quencher dye (=dual-label probe). Suitable fluorescent reporter and quencher dyes/moieties are known to a person skilled in the art and include, but are not limited to the reporter dyes/moieties 6-FAM™, JOE™, Cy5®, Cy3® and the quencher dyes/moieties dabcyl, TAMRA™, BHQ™-1, -2 or -3. Preferably primers for use in accordance with the present invention have a length of 15 to 30 nucleotides, and are in particular deoxyribonucleotides. In one embodiment, the primers are designed so as to (1) be specific for the target mRNA-sequence of as HLA gene or being derived therefrom, (2) provide an amplicon size of less than 120 bp (preferably less than 100 bp), (3) be mRNA-specific (consideration of exons/introns; preferably no amplification of genomic DNA), (4) have no tendency to dimerize and/or (5) have a melting temperature Tₘ in the range of from 58° C. to 62° C. (preferably, Tₘ is approximately 60° C.). As mentioned, the probe is required for a RT-qPCR according to (2) but the probe can be replaced by an intercalating dye in the case of a RT-qPCR according to (1), such as SYBR green.

As one alternative of qPCR also electrophoretic techniques or as one further alternative a DNA microarray may be used to obtaining the levels of the nucleic acid molecule of the first aspect of the invention. The conventional approach to mRNA identification and quantitation is through a combination of gel electrophoresis, which provides information on size, and sequence-specific probing. The Northern blot is the most commonly applied technique in this latter class. The ribonuclease protection assay (RPA) was developed as a more sensitive, less labor-intensive alternative to the Northern blot. Hybridization is performed with a labeled ribonucleotide probe in solution, after which non-hybridized sample and probe are digested with a mixture of ribonucleases (e.g., RNase A and RNase T1) that selectively degrade single-stranded RNAs. Subsequent denaturing polyacrylamide gel electrophoresis provides a means for quantitation and also gives the size of the region hybridized by the probe. For both Northern blot and RPA, the accuracy and precision or quantitation are functions of the detection method and the reference or standard utilized. Most commonly, the probes are radiolabeled with 32P or 33P, in which case the final gel is exposed to X-ray film or phosphor screen and the intensity of each band quantified with a densitometer or phosphor imager, respectively. In both cases, the exposure time can be adjusted to suit the sensitivity required, but the phosphor-based technique is generally more sensitive and has a greater dynamic range. As an alternative to using radioactivity, probes can be labeled with an antigen or hapten, which is subsequently bound by a horseradish peroxidase- or alkaline phosphatase-conjugated antibody and quantified after addition of substrate by chemiluminescence on film or a fluorescence imager. In all of these imaging applications, subtraction of the background from a neighboring region of the gel without probe should be performed. The great advantage of the gel format is that any reference standards can be imaged simultaneously with the sample. Likewise, detection of a housekeeping gene is performed under the same conditions for all samples.

In addition, next generation sequencing (NGS) may be used (Behjati and Tarpey, Arch Dis Child Educ Pract Ed. 2013 December; 98(6): 238). NGS is a RNA or DNA sequencing technology which has revolutionised genomic research. Using NGS an entire human genome can be sequenced within a single day. In contrast, the previous Sanger sequencing technology, used to decipher the human genome, required over a decade to deliver the final draft. In view of the present invention NGS could be used to quantify in open configuration (genome wide exome sequencing) or as focussed panel harbouring the respective HLA genes and isoforms disclosed in this application.

For the construction of DNA microarrays two technologies have emerged. Generally, the starting point in each case for the design of an array is a set of sequences corresponding to the genes or putative genes to be probed. In the first approach, oligonucleotide probes are synthesized chemically on a glass substrate. Because of the variable efficiency of oligonucleotide hybridization to cDNA probes, multiple oligonucleotide probes are synthesized complementary to each gene of interest. Furthermore, for each fully complementary oligonucleotide on the array, an oligonucleotide with a mismatch at a single nucleotide position is constructed and used for normalization. Oligonucleotide arrays are routinely created with densities of about 10⁴-10⁶ probes/cm². The second major technology for DNA microarray construction is the robotic printing of cDNA probes directly onto a glass slide or other suitable substrate. A DNA clone is obtained for each gene of interest, purified, and amplified from a common vector by PCR using universal primers. The probes are robotically deposited in spots on the order of 50-200 μm in size. At this spacing, a density of, for example, approximately 10³ probes/cm² can be achieved.

Levels of the protein or peptide may be determined, for example, by using a “molecule binding to the protein or peptide” and preferably a “molecule specifically binding to the protein or peptide”. A molecule binding to the protein or peptide designates a molecule which under known conditions occurs predominantly bound to the protein or peptide. A “molecule binding to the protein or peptide” may be one of the herein below described binding molecules, preferably inhibitors of the protein or peptide, such as antibodies, aptamers, etc. Levels of the protein or peptide may also be obtained by using Western Blot analysis, mass spectrometry analysis, FACS-analysis, ELISA, and immunohistochemistry. These techniques are non-limiting examples of methods which may be used to qualitatively, semi-quantitatively and/or quantitatively detect a protein or peptide.

Western blot analysis is a widely used and well-know analytical technique used to detect specific proteins or peptides in a given sample, for example, a tissue homogenate or body extract. It uses gel electrophoresis to separate native or denatured proteins or peptides by the length of the (poly)peptide (denaturing conditions) or by the 3-D structure of the protein (native/non-denaturing conditions). The proteins or peptides are then transferred to a membrane (typically nitrocellulose or PVDF), where they are probed (detected) using antibodies specific to the target protein.

Also mass spectrometry (MS) analysis is a widely used and well-know analytical technique, wherein the mass-to-charge ratio of charged particles is measured. Mass spectrometry is used for determining masses of particles, for determining the elemental composition of a sample or molecule, and for elucidating the chemical structures of molecules, such as proteins, peptides and other chemical compounds. The MS principle consists of ionizing chemical compounds to generate charged molecules or molecule fragments and measuring their mass-to-charge ratios.

Fluorescence activated cell sorting (FACS) analysis is a widely used and well-known analytical technique, wherein biological cells are sorted based upon the specific light scattering of the fluorescent characteristics of each cell. Cells may be fixed in 4% formaldehyde, permeabilized with 0.2% Triton-X-100, and incubated with a fluorophore-labeled antibody (e.g. mono- or polyclonal anti-HLA antibody).

Enzyme-linked immunosorbent assay (ELISA) is a widely used and well-know sensitive analytical technique, wherein an enzyme is linked to an antibody or antigen as a marker for the detection of a specific protein or peptide.

Immunohistochemistry (IHC) is the most common application of immunostaining. It involves the process of selectively identifying antigens (proteins) in cells or a tissue section by exploiting the principle of antibodies binding specifically to antigens in biological tissues. In combination with particular devices IHC can be used for quantitative in situ assessment of protein expression (for review Cregger et al. (2006) Arch Pathol Lab Med, 130:1026-1030). Quantitative IHC takes advantage of the fact that staining intensity correlates with absolute protein levels.

Methods for determining whether a subject responded to one or more of the tumor therapies and also for determining whether a subject that did respond to one or more of the tumor therapies are well-known in the art. Generally a tumor patient responds to a therapy if the tumor shrinks (in case of a solid tumor), if the number of tumor cells in a non-solid tumor (such as a blood cancer) or If the symptoms conferred by the tumorous disease are reduced or stay the same (“stabilizes”). Generally a tumor patient does not respond if the tumor worsens (e.g. Increases it's size, increases its number of cells or in case the symptoms conferred by the tumorous disease aggravate) during treatment. In the connection with a response it is preferred that the tumor shrinks.

The definitive proof of the effectiveness of a therapy is improvement in clinical symptoms and survival whereas the definitive proof of the non-effectiveness of a therapy is worsening of clinical symptoms and ultimately the death of the subject. As part of this invention the disease specific survival is frequently being used, which is defined by the start of the treatment option under investigation until cancer specific death. Imaging, in particular of tumor lesions, is generally used to assess therapeutic effects earlier. Current response assessment is based primarily on changes in tumor size as measured by CT (computer tomography) or other anatomic imaging modalities, wherein shrinkage of the tumor size indicates a response. Also, imaging of tumor metabolism with PET (positron-emission-tomography) and the glucose analog ¹⁸F-FDG represents an attractive approach for assessing the effects of therapy objectively and quantitatively.

With respect to the evaluation of solid tumors it is preferred to use the response evaluation criteria in solid tumors (RECIST). RECIST is a set of rules that define when tumors in tumor patients ameliorate, stay the same, or worsen during treatment. The criteria were published in February 2000 by an international collaboration including the European Organisation for Research and Treatment of Cancer (EORTC), National Cancer Institute of the United States, and the National Cancer Institute of Canada Clinical Trials Group. Today, the majority of clinical trials evaluating cancer treatments for objective response in solid tumors use RECIST. These criteria were updated in 2009. With respect to the valuation of solid tumors it is also preferred to use the PET response criteria in solid tumors (PERCIST). PERCIST is an alternative set of rules that define when tumors in tumor patients ameliorate, stay the same, or worsen during treatment, using positron-emission-tomography (PET). These criteria were established in 2009.

The one or more subjects that responded or not responded, respectively, are with increasing preference at least 2, at least 5, at least 10 subjects, at least 25 subjects, and at least 50 subjects. Taking more than one subject has the advantage to bias for level differences among the patients with a response or no response, respectively.

Predetermined standards designate previously obtained values from one or more subjects that responded to one or more of the tumor therapies or one or more subjects that did not respond to one or more of the tumor therapies.

The Increased level(s) of (B) and (B) are with increasing preference at least 1.1-fold, 1.2-fold, 1.3-fold, 1.4-fold, 1.5-fold, 2-fold, 3-fold, 4-fold increased as compared to the level of (A). The decreased level(s) of (B) and (B′) are with increasing preference at least at least 1.1-fold, 1.2-fold, 1.3-fold, 1.4-fold, 1.5-fold, 2-fold, 3-fold, 4-fold decreased as compared to the level of (A). Substantially the same level(s) of (B) and (B′) preferably differ (i.e. higher or lower) by less than 10%, more preferably less that 5% from the control or predetermined standard. For example, if the level in (A) Is set to 100%, a substantially same level may be between less than 110% and more than 90% of the 100% control level.

As can be taken, from the examples herein below in was surprisingly found that the high expression level of membrane-bound HLA-G (exon 8 probe), soluble or membrane bound HLA-G (exon 3 probe), membrane-bound HLA-L (exon 7 probe), soluble HLA-H (exon 2/3 probe) and soluble HLA-J (exon 4/5 probe) in patients having bladder cancer and undergoing immune checkpoint therapy (anti-PD-1 or anti-PDL-1) is adversely associated with the survival of these patients. The higher the expression level of these HLA genes the more likely the patients died from the cancer within 2 years. It has to be taken into account, that posttranscriptional events may affect the membrane bound HLA isoforms. Therefore the determination of membrane bound HLA-G mRNA isoforms determined by exon 8 quantification may after proteolytic cleavage events following translation into the protein structures ultimatively result in soluble fragments of biological activity. However, the HLA mRNA expression levels were measured in tumor tissue samples that were obtained from the bladder cancer patients before the start of the immune checkpoint therapy. Hence, the data in the examples show that the subject's expression levels of the HLA-G, L, H and J genes or proteins can be used in order predict before an immune checkpoint therapy is started whether the subject will likely benefit from the treatment or not. While low expression levels are associated with superior disease specific survival high expression levels are associated with inferior disease specific survival.

It is believed that the predictive value of HLA-G, L, H and J expression levels shown in the examples for the survival of bladder cancer patients under immune checkpoint therapy is also applicable to other tumors and anti-tumor treatments, e.g. immunotherapies in general, chemotherapy, anti-hormonal therapy and anti-tyrosin therapy. This is because it can be assumed that high HLA-G, L, H and J expression levels help the tumor cells or a subpopulation of the tumor cells to escape the anti-tumor therapy, as any effective anti-cancer therapy results in tumor cell destruction and exposition of antigens to the immune system thereby demasking the tumor. Cellular strategies to reduce immune recognition as being conferred by HLA-G, L, H and J expression, therefore are of general importance not only for immune therapies but also for chemotherapy and/or anti-hormonal and/or tyrosin-kinase inhibitory therapy or any therapeutic combination thereof.

With regard to the sequences of membrane-bound human HLA-L, and soluble HLA-H, HLA-J and HLA-L it is of further note that it was surprisingly found herein that HLA-L, HLA-H and HLA-J were erroneously annotated as pseudogenes in the art. In fact, these genes are protein-coding and the expression of HLA-L, HLA-H and HLA-J can be detected in various cancers as is Illustrated in the appended examples. Since HLA-L, HLA-H and HLA-J all were erroneously annotated in the art, HLA-L, HLA-H and HLA-J may be collectively described as a new HLA-group. In addition, the examples herein below show that high expression level of HLA-L, HLA-H and HLA-J in patients having bladder cancer is adversely associated with the survival of these patients. The higher the expression level of these HLA genes, the more likely the patients died from the cancer within 2 years. This body of evidence shows that the expression of HLA forms L, H and J is likely used by tumors as a mechanism of evading the immune system of the tumor patient. These genes and the encoded protein have a function and are not pseudogenes not encoding any functional protein.

In a preferred embodiment or the first aspect of the invention any one of SEQ ID NOs 1 to 6 is any one of SEQ ID NOs to 6, preferably SEQ ID NO: 4 or 5, and any one of SEQ ID NOs 7 to 12 is any one of SEQ ID NOs 9 to 12, preferably SEQ ID NO: 11 or 12.

SEQ ID NOs 9 and 10 are the nucleic acid sequences encoding the soluble HLA forms of membrane-bound HLA-G and HLA-L, and SEQ ID NOs 11 and 12 are soluble HLA-H and HLA-J. SEQ ID NOs 3 to 6 are the corresponding amino acid sequences.

The data in the examples demonstrate on the basis of the HLA classes G, H, L and J that HLA genes and proteins can predict the response of a tumor patient to a tumor therapy as defined herein.

In a preferred embodiment or the first aspect of the invention, the method further comprises determining the mRNA expression level or the protein level or one or more selected from ErbB2, EGFR, CD20, CTLA4, IDO1, LAG3, TIM3, TIM-4, CXCL9, CXCL13, TIGIT, BTLA, CD137, OX40, VISTA, B7-H7, CD27, GITR, TGF-ß Signaling pathway, IL-15, PD-1 and PD-L1, preferably of PD-1 or PD-L1.

In connection with this preferred embodiment it is to be understood that the mRNA expression level(s) or the protein level(s) are to be determined in the subject and are then compared to the respective control or predetermined standard from the known responders or non-responders and/or known survivors or non-survivors, just as explained herein above in connection with the HLA genes.

The mRNA expression level or the protein level of one or more selected from ERBB2. EGFR, CD20, CTLA4, IDO1, LAG3, TIM3, TIM-4, CXCL9, CXCL13, TIGIT, BTLA, CD137, OX40, VISTA, B7-H7, CD27, GITR, TGF-ß Signaling pathway, IL-15, PD-1 and PD-L1, preferably of PD-1 or PD-L1 are alone not of sufficient predictive value for determining whether a subject is likely to respond or not respond to a tumor therapy as defined herein, in particular an immune therapy and more particularly an checkpoint therapy. They may be useful in combination with the method of the present invention.

Thus, the additional analysis of one or more of these level(s) is expected to further improve the predictive value of the method of the invention.

PD-1 (Programmed cell death protein 1, also known as CD279) is a protein on the surface of cells that has a role in regulating the immune system's response to the cells of the human body by down-regulating the immune system and promoting self-tolerance via suppressing T cell inflammatory activity.

PD-L1 (Programmed death-ligand 1, also known as CD274 or 67-H1) is a 40 kDa type 1 transmembrane protein that has been speculated to play a major role in suppressing the immune system during particular events such as pregnancy, tissue allografts, autoimmune disease and other disease states such as hepatitis. Upregulation of PD-L1 may allow cancers to evade the host immune system. Importantly, PD-L1 might be expressed by tumour or non tumour cells such as macrophages etc.

ErbB2 (Receptor tyrosine-protein kinase erbB-2, also known as CD340 or proto-oncogene Neu) is a member of the human epidermal growth factor receptor (HER/EGFR/ERBB) family. Amplification or over-expression of this oncogene has been shown to play an important role in the development and progression of certain aggressive types of breast cancer.

EGFR (epidermal growth factor receptor, also known as HER1) is a transmembrane protein that is a receptor for members of the epidermal growth factor family (EGF family) of extracellular protein ligands.

CD20 is an activated-glycosylated phosphoprotein expressed on the surface of all B-cells beginning at the pro-B phase (CD45R+, CD117+) and progressively increasing in concentration until maturity. CD20 is the target of the monoclonal antibodies rituximab, ocrelizumab, obinutuzumab, ofatumumab, ibritumomab tiuxetan, tositumomab, and ublituximab, which are all active agents in the treatment of all B cell lymphomas, leukemias, and B cell-mediated autoimmune diseases.

CTLA4 (cytotoxic T-lymphocyte-associated protein 4, also known as CD152), is a protein receptor that, functioning as an immune checkpoint (or checkpoint inhibitor), downregulates immune responses. CTLA4 is constitutively expressed in regulatory T cells but only upregulated in conventional T cells after activation—a phenomenon which is particularly notable in cancers.

IDO1 (Indoleamine-pyrrole 2,3-dioxygenase) Is a heme-containing enzyme. IDO1 has been implicated in immune modulation through its ability to limit T-cell function and engage mechanisms of immune tolerance. IDO becomes activated during tumor development, helping malignant cells to escape eradication by the immune system.

LAG3 (Lymphocyte-activation gene 3, also known as CD223) is a cell surface molecule with diverse biologic effects on T cell function. It is an immune checkpoint receptor and as such is the target of various drug development programs by pharmaceutical companies seeking to develop new treatments for cancer and autoimmune disorders.

TIM-3 (T-cell immunoglobulin and mucin-domain containing-3, also know as Hepatitis A virus cellular receptor 2 (HAVCR2)) mediates the CD8+ T-cell exhaustion. TIM-3 has also been shown as a CD4+Th1-specific cell surface protein that regulates macrophage activation and enhances the severity of experimental autoimmune encephalomyelitis in mice.

TIM-4 (T-cell immunoglobulin and mucin-domain containing-4) is a phosphatidylserine receptor that enhances the engulfment of apoptotic cells. TIM-4 is involved in regulating T-cell proliferation and lymphotoxin signaling.

CXCL9 (chemokine (C-X-C motif) ligand 9) is a small cytokine belonging to the CXC chemokine family that is also known as Monokine induced by gamma interferon (MIG). CXCL9 is a T-cell chemoattractant, which is induced by IFN-γ.

CXCL13 (chemokine (C-X-C motif) ligand 1, also known as B lymphocyte chemoattractant (BLC) or B cell-attracting chemokine 1 (BCA-1)) is a small chemokine belonging to the CXC chemokine family. As its name suggests, this chemokine is selectively chemotactic for B cells belonging to both the B-1 and B-2 subsets, and elicits its effects by interacting with chemokine receptor CXCR5.

TIGIT also called T cell immunoreceptor with Ig and ITIM domains) is an immune receptor present on some T cells and Natural Killer Cells (NK). It is also identified as WUCAM and Vstm3. TIGIT and PD-1 have been shown to be overexpressed on tumor antigen-specific (TA-specific) CD8+ T cells and CD8+ tumor infiltrating lymphocytes (TILs) from individuals with melanoma.

BTLA (B- and T-lymphocyte attenuator, also known as CD272) expression is induced during activation of T cells, and BTLA remains expressed on Th1 cells but not Th2 cells. BTLA activation inhibits the function of human CD8+ cancer-specific T cells.

CD137 is also known as tumor necrosis factor receptor superfamily member 9 (TNFRSF9), 4-1BB and induced by lymphocyte activation (ILA). The best characterized activity of CD137 is its costimulatory activity for activated T cells. Crosslinking of CD137 enhances T cell proliferation, IL-2 secretion, survival and cytolytic activity. Further, it can enhance immune activity to eliminate tumors.

Ox40 (also known as tumor necrosis factor receptor superfamily, member 4 (TNFRSF4) and CD134) is a secondary co-stimulatory immune checkpoint molecule, expressed after 24 to 72 hours following activation; its ligand, OX40L, is also not expressed on resting antigen presenting cells, but is following their activation. Expression of OX40 is dependent on full activation of the T cell; without CD28, expression or OX40 Is delayed and of fourfold lower levels.

VISTA (V-domain Ig suppressor of T cell activation) Is a type I transmembrane protein that functions as an immune checkpoint. VISTA can act as both a ligand and a receptor on T cells to inhibit T cell effector function and maintain peripheral tolerance.

B7-H7 (also known as human endogenous retrovirus-H long terminal repeat associating 2 (HHLA2)) is a B7 family member that regulates human T-cell functions. B7-H7 was previously known as with unidentified function. B7-H7 has been identified as a specific ligand for human CD28H. The B7-H7-CD28H pathway strongly promoted CD4+ T-cell proliferation and cytokine production via an AKT-dependent signaling cascade in the presence or TCR signaling, suggesting B7-H7 comprises a new co-stimulatory pathway. The first IgV domain of B7-H7, which presumably binds to a putative receptor, shows the highest homology to other B7 family members.

CD27 is required for generation and long-term maintenance of T cell immunity. It binds to ligand CD70, and plays a key role in regulating B-cell activation and immunoglobulin synthesis.

GITR (glucocorticoid-induced TNFR-related protein, also known as tumor necrosis factor receptor superfamily member 18 (TNFRSF18) and activation-inducible TNFR family receptor (AITR)) has been shown to have increased expression upon T-cell activation, and it is thought to play a key role in dominant immunological self-tolerance maintained by CD25+/CD4+ regulatory T cells. Knockout studies in mice also suggest the role of this receptor is in the regulation of CD3-driven T-cell activation and programmed cell death.

The transforming growth factor beta (TGFβ) signaling pathway is involved in many cellular processes in both the adult organism and the developing embryo including cell growth, cell differentiation, apoptosis, cellular homeostasis and other cellular functions. In spite of the wide range of cellular processes that the TGFβ signaling pathway regulates, the process is relatively simple. TGFβ superfamily ligands bind to a type receptor, which recruits and phosphorylates a type I receptor. The type I receptor then phosphorylates receptor-regulated SMADs (R-SMADs) which can now bind the coSMAD SMAD4. R-SMAD/coSMAD complexes accumulate in the nucleus where they act as transcription factors and participate in the regulation of target gene expression.

IL-15 (Interleukin-15) is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells.

The present invention relates in a second aspect to a binding molecule, preferably an inhibitor of at least one nucleic acid molecule as defined in connection with the first aspect of the invention or at least one protein or peptide as defined in connection with the first aspect of the invention for use in the treatment of a tumor in a subject, wherein the inhibitor is to be used in combination with (i) an immunotherapy; (ii) a chemotherapy; (iii) an anti-hormonal therapy; and/or (iv) an anti-tyrosin kinase therapy.

The definitions provides herein above with the first aspect of the invention apply mutatis mutandis to the second aspect of the invention.

The binding molecule, preferably inhibitor of a nucleic acid molecule as defined in connection of the first aspect of the invention is preferably selected from a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease. Further details on these classes will be provided herein below.

The binding molecule, preferably the inhibitor of the HLA protein according to the invention is preferably selected from a small molecule, an antibody or antibody mimetic, and an aptamer, wherein the antibody mimetic is preferably selected from affibodies, adnectins, anticalins. DARPins, avimers, nanofitins, affilins, kunitz domain peptides, Fynomers®, trispecific binding molecules and probodies.

As used herein, the term “antibody mimetics” refers to compounds which, like antibodies, can specifically bind antigens, such the HLA proteins of SEQ ID NOs 1 to 6 in the present case, but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunltz domain peptides and Fynomers®. These polypeptides are well known in the art and are described in further detail herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10th extracellular domain of human fibronectin III (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity, i.e. against a HLA protein, can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci USA. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (H) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding or target molecules occurs via the A-domain and domains with the desired binding specificity, i.e. for a HLA protein, can be selected, for example, by phage display techniques. The binding specificity of the different A-domains contained in an avimer may, but does not have to be identical (Weidle U H, et al., (2013). Cancer Genomics Proteomics; 10(4):155-68).

A “nanofitin” (also known as affitin) Is an antibody mimetic protein that is derived from the DNA binding protein Sac7d of Sulfolobus acidocaldarius. Nanofitins usually have a molecular weight of around 7 kDa and are designed to specifically bind a target molecule, such as e.g. a HLA protein, by randomising the amino acids on the binding surface (Mouratou B, Behar G. Paillard-Laurance L, Colinet S. Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity, i.e. against a HLA gene in accordance with the invention, is effected, for example, by phage display or ribosome display techniques. Depending on the scaffold, affilins have a molecular weight of approximately 10 or kDa. As used herein, the term affilin also refers to di- or multimerised forms of affilins (Weidle, et al., (2013), Cancer Genomics Proteomics; 10(4):155-48).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway Inhibitor (TFPI). Kunitz domains have a molecular weight of approximately 6 kDA and domains with the required target specificity, i.e. against a HLA protein, can be selected by display techniques such as phage display (Weidle et al., (2013), Cancer Genomics Proteomics: 10(4):155-68).

As used herein, the term “Fynomer®” refers to a non-immunoglobulin-derived binding polypeptide derived from the human Fyn SH3 domain. Fyn SH3-derived polypeptides are well-known in the art and have been described e.g. In Grabulovski et al. (2007) JBC, 282, p. 3196-3204, WO 2008/022759. Bertschinger et al (2007) Protein Eng Des Sel 20(2):57-88. Gebauer and Skerra (2009) Curr Opinion in Chemical Biology 13:245-255, or Schlatter et al. (2012), MAbs 4:4, 1-12).

The term “trispecific binding molecule” as used herein refers to a polypeptide molecule that possesses three binding domains and is thus capable of binding, preferably specifically binding to three different epitopes. At least one of these three epitopes is an epitope of the HLA protein in accordance with the invention. The two other epitopes may also be epitopes of a HLA protein in accordance with the invention or may be epitopes of one or two different antigens. The trispecific binding molecule is preferably a TriTac. A TriTac is a T-cell engager for solid tumors which comprised of three binding domains being designed to have an extended serum half-life and be about one-third the size of a monoclonal antibody.

As used herein, the term “probody” refers to a protease-activatable antibody prodrug. A probody consists of an authentic IgG heavy chain and a modified light chain. A masking peptide is fused to the light chain through a peptide linker that is cleavable by tumor-specific proteases. The masking peptide prevents the probody binding to healthy tissues, thereby minimizing toxic side effects. For example, in a probody a small molecule, antibody or protein drug or aptamer may be bound to a masking peptide which limits or prevents binding to the HLA protein in accordance with the invention and which masking peptide can be cleaved by a protease. Proteases are enzymes that digest proteins into smaller pieces by cleaving specific amino acid sequences known as substrates. In normal healthy tissue, protease activity is tightly controlled. In cancer cells, protease activity is upregulated. In healthy tissue or cells, where protease activity is regulated and minimal, the target-binding region of the probody remains masked and is thus unable to bind. On the other hand, in diseased tissue or cells, where protease activity is upregulated, the target-binding region of the probody gets unmasked and is thus able to bind and/or inhibit.

A binding molecule of the second aspect is a compound being capable of binding to the nucleic acid molecule, protein or peptide as defined herein. The binding molecule preferably specifically binds to the nucleic acid molecule, protein or peptide. Specific binding designates that the binding molecule essentially does not or essentially does not bind to other nucleic acid molecules, proteins or peptides than the nucleic acid molecule, protein or peptide as defined herein. In particular, it is preferred that the binding molecule is not capable to bind to other HLA proteins than the respective selected HLA protein. A binding molecule of the invention is, for example, suitable for research or diagnostic purposes. For example, an antibody binding to the protein according to the invention can be used in immuonassays, such as an ELISA or Western Blot. Immunoassays are biochemical tests that can measure the presence or concentration the protein of the second aspect in a sample (e.g. a solution). In addition the antibody may be used for tissue or cellular stainings including but not limited to such as IHC, FACS, Immunefluorescent methods etc. The binding molecule of the protein of the second aspect is preferably capable of inhibiting the nuclei acid molecule, protein or peptide as defined herein. In this case the binding molecule is designated inhibitor.

A compound inhibiting the expression of the nucleic acid molecule and/or the protein according to the invention is in accordance with the present invention (i) a compound lowering or preventing the transcription of the gene encoding the nucleic acid molecule and/or the protein according to the invention, or (ii) is a compound lowering or preventing the translation of the mRNA encoding the protein according to the invention. Compounds of (i) include compounds interfering with the transcriptional machinery and/or its interaction with the promoter of said gene and/or with expression control elements remote from the promoter such as enhancers. Compounds of (ii) include compounds interfering with the translational machinery. The compound inhibiting the expression of the nucleic acid molecule and/or the protein according to the invention specifically inhibits the expression of the nucleic acid molecule and/or the protein according to the invention, for example, by specifically interfering with the promoter region controlling the expression. Preferably, the transcription of the nucleic acid molecule and/or the protein according to the invention or the translation of the protein according to the invention is reduced with increasing preference by at least 10%, at least 20%, at least 30%, at least 50%, at least 75% such as at least 90% or 95%, at least 98% and most preferred by about 100% (e.g., as compared to the same experimental set up in the absence of the compound).

A compound inhibiting the activity of the nucleic acid molecule, protein and/or the protein according to the invention in accordance with the present invention causes said nucleic acid molecule, peptide and/or protein to perform its/their function with lowered efficiency. The compound inhibiting the activity of the nucleic acid molecule, peptide and/or the protein according to the invention specifically inhibits the activity of said nucleic acid molecule, peptide and/or protein. As will be further detailed herein below, the compound inhibiting the activity or the nucleic acid molecule, peptide and/or the protein according to the invention may specifically inhibit the activity of said nucleic acid molecule, peptide and/or protein by interacting with the nucleic acid molecule, peptide and/or protein itself or by specifically inhibiting (preferably killing) cells that produce said nucleic acid molecule, peptide and/or said protein and/or bind to said peptide or protein. Preferably, the activity of the nucleic acid molecule, peptide and/or the protein according to the invention is reduced by at least 50%, more preferred at least 75% such as at least 90% or 95%, even more preferred at least 98%, and most preferably about 100% (e.g., as compared to the same experimental set up in the absence of the compound).

As an alternative option a compound inhibiting the activity of said nucleic acid molecule, protein and/or the protein in accordance with the present invention also comprises nucleic acids or analogous thereof that are used to vaccinate the patient against specific HLA isoforms. The process of vaccination may be based on RNA, protein or peptide level requiring additional modifications for stabilization within the in vivo situation in the human body. Such method could be adopted from personalized mutanome vaccination approaches (Sahin U. Personalized RNA vaccines mobilizes poly-specific therapeutic immunity against cancer. Nature 2017).

As a further option a compound inhibiting the activity of said nucleic acid molecule, protein and/or the protein according to the invention also comprises isolation of naturally occurring auto-antibodies or cells producing naturally occurring auto-antibodies against respective HLA genes, isoforms and fragments, that could be modified or propagated before reintroduction into the respective patients.

The activity of the nucleic acid molecule, peptide and/or the protein according to the invention Is in accordance with this invention preferably its/their capability to induce resistance to a tumor therapy as defined herein above in cancer patients. Means and methods for determining this activity are established in the art and are illustrated in the examples herein below. In accordance with the medical aspects of the invention, this activity of the nucleic acid molecule and/or the protein according to the invention is therefore to be inhibited.

The efficiency of inhibition by an inhibitor can be quantified by methods comparing the level of activity in the presence of the inhibitor to that in the absence of the inhibitor. For example, the change in the amount of the nucleic acid molecule and/or the protein according to the invention formed may be used in the measurement. The efficiency of several inhibitors may be determined simultaneously in high-throughput formats. High-throughput assays, independently of being biochemical, cellular or other assays, generally may be performed in wells of microtiter plates, wherein each plate may contain 96, 384 or 1538 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within a short time, mixtures of, for example 10, 20, 30, 40, 50 or 100 test compounds may be added to each well. In case a well exhibits the expected activity, said mixture of test compounds may be de-convoluted to identify the one or more test compounds in said mixture giving rise to said activity.

The compounds inhibiting the expression and/or the activity of the nucleic acid molecule and/or the protein according to the invention may be formulated as vesicles, such as liposomes or exososmes. Liposomes have attracted great interest because of their specificity and the duration of action they offer from the standpoint of drug delivery. Liposomal cell-type delivery systems have been used to effectively deliver nucleic acids, such as siRNA in vivo into cells (Zimmermann et al. (2006) Nature, 441:111-114). Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are phagocytosed by macrophages and other cells in vivo. Exosomes are lipid packages which can carry a variety of different molecules including RNA (Alexander et al. (2015), Nat Commun; 6:7321). The exosomes including the molecules comprised therein can be taken up by recipient cells. Hence, exosomes are important mediators of intercellular communication and regulators of the cellular niche. Exosomes are useful for diagnostic and therapeutic purposes, since they can be used as delivery vehicles, e.g. for contrast agents or drugs.

The compounds inhibiting the expression and/or the activity of the nucleic acid molecule, peptide and/or the protein according to the invention can be administered to the subject at a suitable dose and/or a therapeutically effective amount. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 mg per day. Furthermore, if for example said compound is an iRNA agent, such as an siRNA, the total pharmaceutically effective amount of pharmaceutical composition administered will typically be less than about 75 mg per kg of body weight, such as for example less than about 70, 60, 50, 40, 30, 20, 10, 5, 2, 1, 0.5, 0.1, 0.05, 0.01, 0.005, 0.001, or 0.0005 mg per kg of body weight. More preferably, the amount will be less than 2000 nmol of iRNA agent (e.g., about 4.4×10¹⁶ copies) per kg of body weight, such as for example less than 1500, 750, 300, 150, 75, 15, 7.5, 1.5, 0.75, 0.15, 0.075, 0.015, 0.0075, 0.0015, 0.00075 or 0.00015 nmol of iRNA agent per kg of body weight. The length of treatment needed to observe changes and the interval following treatment for responses to occur vary depending on the desired effect. The length of treatment needed to observe changes and the interval following treatment for responses to occur vary depending on the desired effect. The particular amounts may be determined by conventional tests which are well known to the person skilled in the art. Suitable tests are, for example, described in Tamhane and Logan (2002), “Multiple Test Procedures for Identifying the Minimum Effective and Maximum Safe Doses of a Drug”, Journal of the American statistical association, 97(457):1-9.

The compounds inhibiting the expression and/or the activity of the nucleic acid molecule, peptide and/or the protein according to the invention are preferably admixed with a pharmaceutically acceptable carrier or excipient to form a pharmaceutical composition. In accordance with the present invention, the term “pharmaceutical composition” relates to a composition for administration to a patient, preferably a human patient. The pharmaceutical composition of the invention comprises the compounds recited above. It may, optionally, comprise further molecules capable of altering the characteristics of the compounds of the invention thereby, for example, stabilizing, modulating and/or activating their function. The composition may be in solid, liquid or gaseous form and may be. Inter ala, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods. Means and methods for preparing pharmaceutical compositions are described, for example, in Formulation tools for Pharmaceutical Development (2005), ISBN-10: 1907568999 or the Handbook of Pharmaceutical Manufacturing Formulations. ISBN-10: 9781420081169.

The pharmaceutical compositions may be administered by any suitable route. The actual route to be selected, for example, depends on physical and chemical properties of the drug, the site of the desired action, the rate of extent of absorption of the drug from different mutes, the metabolism of the drug, and the condition of the patient. Examples of administration routes are enteral/gastrointestinal, topical and parenteral. In addition the pharmaceutical compositions may be applied as instillation therapy into the bladder in case of bladder cancer or neoplastic lesions thereof. The administration as instillation therapy is regarded as part of the invention particularly for the combination of immunological, chemotherapeutic, anti-hormonal or anti-tyrosin kinase compounds together with anti-HLA agents as described as part of this application.

These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently.

As discussed above, the data in the examples show that high expression levels of these HLAs are associated with inferior disease specific survival. Moreover, it can be assumed that high HLA-G, L, H and J expression levels help the tumor cells or a subpopulation of the tumor cells to escape the anti-tumor therapy.

It is therefore also assumed that a combination therapy, wherein a classical anti-tumor treatment (e.g. (i) an immunotherapy; (ii) a chemotherapy; (iii) an anti-hormonal therapy; and/or (iv) an anti-tyrosin kinase therapy) is combined with an inhibitor of HLA-G, L, H or J further improves the anti-tumor treatment. Such combined anti-tumor treatment may be done as a precautionary measure and in particular should de done in patients that were diagnosed by the method of the invention as expressing high levels of HLA-G, L, H and/or J at the outset of the therapy. In such patients a treatment failure could be turned into treatment success.

In a preferred embodiment of the second aspect of the invention, the subject has been predicted to not respond to (i) an immunotherapy; (II) a chemotherapy; (iii) an anti-hormonal therapy; and/or (v) an anti-tyrosin kinase therapy by the method of the first aspect of the invention.

The prior diagnosis of the of the subject to be treated as to not respond to (i) an immunotherapy; (ii) a chemotherapy; (iii) an anti-hormonal therapy; and/or (iv) an anti-tyrosin kinase therapy indicates the necessity to treat the subject in addition by a binding molecule, preferably an inhibitor of the invention.

This is because the expression of the HLA genes as discussed herein above is believed to protect the malignant cells in the subject from (i) an immunotherapy; (i) a chemotherapy; (iii) an anti-hormonal therapy; and/or (iv) an anti-tyrosin kinase therapy, so that a combination of the binding molecule, preferably an inhibitor of the invention with (i) an immunotherapy; (ii) a chemotherapy; (iii) an anti-hormonal therapy; and/or (iv) an anti-tyrosin kinase therapy is capable to turn the (expected) treatment failure into a treatment success.

In a preferred embodiment of the second aspect of the invention, the Inhibitor is a small molecule inhibitor, a nucleotide-based inhibitor or an amino acid-based inhibitor.

The “small molecule” as used herein is preferably an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. The organic molecule is preferably an aromatic molecule and more preferably a heteroaromatic molecule. In organic chemistry, the term aromaticity is used to describe a cyclic (ring-shaped), planar (flat) molecule with a ring of resonance bonds that exhibits more stability than other geometric or connective arrangements with the same set of atoms. Aromatic molecules are very stable, and do not break apart easily to react with other substances. In a heteroaromatic molecule at least one of the atoms in the aromatic ring Is an atom other than carbon, e.g. N, S, or O. For all above-described organic molecules the molecular weight is preferably in the range of 200 Da to 1500 Da and more preferably in the range of 300 Da to 1000 Da.

Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small inorganic molecule has a molecular weight of less than about 2000 Da, or less than about 1000 Da such as less than about 500 Da, and even more preferably less than about 250 Da. The size of a small molecule can be determined by methods well-known in the art, e.g., mass spectrometry. The small molecules may be designed, for example, based on the crystal structure of the target molecule, where sites presumably responsible for the biological activity can be identified and verified in in vivo assays such as in vivo high-throughput screening (HTS) assays.

A nucleotide-based inhibitor comprises or consists of a nucleic acid sequence. The nucleotide-based inhibitor may comprise or consist of RNA, DNA or both. The nucleotide-based or nucleotide-analog based inhibitor of the invention is a molecule that binds specifically to an HLA gene of SEQ ID NOs 7 to 12 and in addition inhibits the activity of the HLA encoded by said gene. As used herein specific binding means that the inhibitor specifically targets the HLA and does substantially not exert any off-target inhibitory effects, in particular on other cellular nucleic acid molecules.

An amino acid-based Inhibitor comprises or consists of an amino acid sequence and preferably an amino acid sequence of at least 25, more preferably at least 50 amino acids. The amino acid-based inhibitor of the invention is a molecule that binds specifically to a HLA of SEQ ID NO 1 to 6 and in addition inhibits the activity of said HLA. The amino acid-based inhibitor preferably comprises natural amino acids but may also comprise unnatural amino acids. The amino acid-based inhibitor is preferably selected or designed such that it specifically binds to an amino acid sequence selected from SEQ ID NOs 1 to 6.

In connection with the second aspect of the invention, the binding molecule, preferably the inhibitor may also be a cell such as a T-cell, wherein the T-cell is preferably a CAR-T-cell.

The cell generally carries on its surface a binding molecule, preferably an inhibitor of at least one nucleic acid molecule in accordance with the invention or at least one protein or peptide in accordance with the invention. In the case of a T-cell the binding molecule, preferably the inhibitor is a naturally occurring or chimeric T-cell receptor that specifically targets at least one protein or peptide in accordance with the invention. Chimeric antigen receptor T-cells (also known as CAR T-cells) are T-cells that have been genetically engineered to produce an artificial T-cell receptor for use in immunotherapy.

Chimeric antigen receptors (CARs, also known as chimeric immunoreceptors, chimeric T cell receptors or artificial T cell receptors) are accordingly receptor proteins that have been engineered to give T-cells the new ability to specifically target at least one protein or peptide in accordance with the invention. The receptors are chimeric because they combine both antigen-binding and T-cell activating functions into a single receptor.

In a more preferred embodiment of the second aspect of the invention, the nucleotide-based inhibitor or amino acid-based inhibitor is an aptamer, a ribozyme, a siRNA, a shRNA or an antisense oligonucleotide, a CRISPR-endonuclease-based construct, a meganuclease, a zinc finger nuclease, or a transcription activator-like (TAL) effector (TALE) nuclease and the amino acid-based inhibitor is an antibody or a protein drug.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications.

Nucleic acid aptamers are nucleic acid species that normally consist of (usually short) strands of oligonucleotides. Typically, they have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers are usually peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which Is a -Cys-Gly-Pro-Cys-loop (SEQ ID NO: 13) in the wild protein, the two cysteins lateral chains being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminatory recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamers' inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

A ribozyme (from ribonucleic acid enzyme, also called RNA enzyme or catalytic RNA) Is an RNA molecule that catalyses a chemical reaction. Many natural ribozymes catalyse either their own cleavage or the cleavage of other RNAs, but they have also been found to catalyse the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron Is an example for larger ribozymes. The principle of catalytic self-cleavage has become well established in recent years. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is Inserted between them. The best results are usually obtained with short ribozymes and target sequences.

A recent development, also useful in accordance with the present invention, is the combination of an aptamer, recognizing a small compound, with a hammerhead ribozyme. The conformational change induced in the aptamer upon binding the target molecule can regulate the catalytic function of the ribozyme.

In accordance with the present Invention, the term “small Interfering RNA (siRNA)”, also known as short Interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is Involved in the RNA Interference (RNAI) pathway where the siRNA Interferes with the expression of a specific gene. In addition to their role in the RNAI pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs Into siRNAs. siRNAs can also be exogenously (artificially) Introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA against the targets in accordance with the invention is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair. 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAI include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of an HLA gene according to the invention after introduction into the respective cells.

The term “antisense nucleic acid molecule”, as used herein, refers to a nucleic acid which is complementary to a target nucleic acid. An antisense molecule in accordance with the Invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

CRISPR/Cas9, as well as CRISPR-Cpf1, technologies are applicable in nearly all cells/model organisms and can be used for knock out mutations, chromosomal deletions, editing of DNA sequences and regulation of gene expression. The regulation of the gene expression can be manipulated by the use of a catalytically dead Cas9 enzyme (dCas9) that is conjugated with a transcriptional repressor to repress transcription a specific gene, here a HLA gene in accordance with the Invention. Similarly, catalytically Inactive, “dead” Cpf1 nuclease (CRISPR from Prevotella and Francisella-1) can be fused to synthetic transcriptional repressors or activators to downregulate endogenous promoters, e.g. the promoter which controls HLA gene expression. Alternatively, the DNA-binding domain of zinc finger nucleases (ZFNs) or transcription activator-like effector nucleases (TALENs) can be designed to specifically recognize a HLA gene or its promoter region or its 5′-UTR thereby Inhibiting the expression of the HLA gene.

Inhibitors provided as Inhibiting nucleic acid molecules that target a HLA gene or a regulatory molecule involved in HLA expression are also envisaged herein. Such molecules, which reduce or abolish the expression of a target HLA or a regulatory molecule include, without being limiting, meganucleases, zinc finger nucleases and transcription activator-like (TAL) effector (TALE) nucleases. Such methods are described in Silva et al., Curr Gene Ther. 2011; 11(1):11-27: Miller et al., Nature biotechnology. 2011; 29(2):143-148, and Klug, Annual review of biochemistry. 2010; 79:213-231.

The term “antibody” as used in accordance with the present Invention comprises, for example, polyclonal or monoclonal antibodies from any species and humanized versions thereof. Furthermore, also derivatives or fragments thereof, which still retain the binding specificity to the target, e.g. the HLA protein of SEQ ID NOs 1 to 6, are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies. A Laboratory Manual”, Cold Spring Harbor Laboratory Press, 198; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigen. The first antigen can be found on the HLA protein in accordance with the Invention. The second antigen may, for example, be a tumor marker that is specifically expressed on cancer cells or a certain type of cancer cells. Non-limiting examples of bispecific antibodies formats are Bicionics (bispecific, full length human IgG antibodies), DART (Dual-affinity Re-targeting Antibody) and BITE (consisting of two single-chain variable fragments (scFvs) of different antibodies) molecules (Kontermann and Brinkmann (2015), Drug Discovery Today, 20(7):838-847).

The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Harlow and Lane (1988) and (1999) and Altshuler et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. In Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Kohler and Milstein, 1975, the trioma technique, the human B-cell hybridoma technique (see e.g. Kozbor D, 1983, Immunology Today, vol. 4:7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal. mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005. Nat Biotechnol., vol. 23(9), 11265). Further, techniques described for the production of single chain antibodies (see, inter alia, U.S. Pat. No. 4,948,778) can be adapted to produce single chain antibodies specific for an epitope of a HLA gene according to the invention. Surface plasmon resonance as employed in the BIAcore system can be used to increase the efficiency of phage antibodies.

As used herein, the term “protein drug” refers to a protein or peptide which displays a therapeutic (either curative or preventive) effect when administered to a subject. Examples of protein drug classes will be discussed herein below.

As discussed, the above-described small molecule, antibody or protein drug and aptamer can specifically bind to the protein according to the invention. This binding may block the immunosuppressive properties of the protein according to the invention and preferably the proteins' capability to induce resistance to a tumor therapy as defined herein in cancer patients and/or to reduce progression free as well as overall survival in cancer patients. In this case the small molecule, antibody or protein drug and aptamer are also referred to as blocking small molecule, antibody or protein drug and aptamer. A blocking small molecule, antibody or protein drug and aptamer blocks Interactions of the HLA protein in accordance with the Invention with other cellular components, such as ligands and receptor which normally Interact with the HLA protein in accordance with the Invention.

The small molecule, antibody or protein drug and aptamer can also be generated in the format of drug-conjugates. In this case the small molecule, antibody or protein drug and aptamer in Itself may not have an Inhibitory effect but the Inhibitory effect is only conferred by the drug. The small molecule, antibody or protein drug and aptamer confer the she-specific binding of the drug to cells producing and/or binding to the HLA protein in accordance with the Invention. The drug is preferably capable to kill cells producing and/or binding to the HLA protein in accordance with the invention. Hence, by combining the targeting capabilities of molecules binding to the HLA protein in accordance with the invention with the cell-killing ability of the drug, the drug conjugates become inhibitors that allow for discrimination between healthy and diseased tissue and cells. Cleavable and non-cleavable linkers to design drug conjugates are known in the art. Non-limiting examples of drugs being capable of killing cells are cytostatic drugs and radioisotopes that deliver radiation directly to the cancer cells.

It is furthermore possible to confine the binding and/or inhibitory activity of the small molecule, antibody or protein drug and aptamer to certain tissues or cell-types, in particular diseased tissues or cell-types. For Instance, probodies may be designed which are further described herein below.

In an even more preferred embodiment of the second aspect of the invention, the protein drug is an antibody mimetic, preferably selected from affibodies, adnectins, anticalins, DARPins, avimers, nanofitins, affilins, Kunitz domain peptides, Fynomers®, trispecific binding molecules and probodies.

In another preferred embodiment of the second aspect of the invention, the nucleotide-based Inhibitor comprises (a) a nucleic acid sequence which comprises or consists of a nucleic acid sequence being complementary to at least 12 continuous nucleotides of a nucleic acid sequence selected from SEQ ID NOs 7 to 12 or a sequence being at least 80% identical thereto, (b) a nucleic acid sequence which comprises or consists of a nucleic acid sequence which is at least 80% identical to the complementary strand of one or more nucleic acid sequences selected from SEQ ID NOs 7 to 12, (c) a nucleic acid sequence which comprises or consists of a nucleic acid sequence according to (a) or (b), wherein the nucleic acid sequence is DNA or RNA. (d) an expression vector expressing the nucleic acid sequence as defined in any one of (a) to (c), preferably under the control of a tumor-specific promoter, or (e) a host comprising the expression vector or (d).

The nucleic acid sequences as defined in items (a) to (c) of this preferred embodiment comprise or consist of sequences being complementary to nucleotides of the HLA gene as defined by one or more of SEQ ID NOs 7 to 12. Hence, the nucleic acid sequences as defined in items (a) to (c) comprise or are antisense nucleic acid sequences.

The nucleic acid sequence according to item (a) of this further preferred embodiment of the invention comprises or consists of a sequence which Is with increasing preference complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides of one or more selected from SEQ ID NOs 7 to 12. These at least 13 nucleotides, at least 14 nucleotides, at least 15 nucleotides, at least 16 nucleotides, at least 17 nucleotides, at least 18 nucleotides, at least 19 nucleotides, at least 20 nucleotides, or at least 21 nucleotides are preferably a contiguous part of one or more selected from SEQ ID NOs 7 to 12, i.e. the nucleotides are consecutive in the respective SEQ ID NO. The format of the nucleic acid sequence according to item (a) is not particularly limited as long as it comprises or consists of at least 12 continuous nucleotides being complementary to a nucleic acid sequence selected from SEQ ID NOs 7 to 12. The nucleic acid sequence according to item (a) comprises or consists of antisense an oligonucleotide. Hence, the nucleic acid sequence according to item (a) reflects the above-mentioned basic principle of the antisense technology which is the use of an oligonucleotide for silencing a selected target RNA through the exquisite specificity of complementary-based pairing. Therefore, it is to be understood that the nucleic acid sequence according to item (a) is preferably in the format of an antisense oligonucleotide or forms part of an siRNA or shRNA as defined herein above. The antisense oligonucleotides are preferably LNA-GapmeRs, AntagomiRs, or antimiRs.

The nucleic acid sequence according to item (b) requiring at least 70% Identity to the complementary strand of one or more nucleic acid sequences selected from SEQ ID NOs 7 to 12 is typically considerably longer than the nucleic acid sequence according to item (a) which comprises an antisense oligonucleotide and comprises at least 12 continuous nucleotides of a nucleic acid sequence selected from SEQ ID NOs 7 to 12. A nucleic acid sequence according to item (b) of the above preferred embodiment of the invention is capable of interacting with, more specifically hybridizing with the target HLA gene. By formation of the hybrid the function of the HLA is reduced or blocked.

The sequence identity of the molecule according to item (b) in connection with a sequence selected from SEQ ID NOs 7 to 12 is with increasing preference at least 75%, at least 80%, at least 85%, at least 90%, at least 92.5%, at least 95%, at least 98%, at least 99% and 100%. The sequence identity in connection with each of SEQ ID NOs 7 to 12 can be individually selected. Means and methods for determining sequence identity are known in the art. Preferably, the BLAST (Basic Local Alignment Search Too) program is used for determining the sequence Identity with regard to one or more of SEQ ID NOs 7 to 12.

In the nucleic acid sequence according to item (c) the nucleotide sequences may be RNA or DNA. RNA or DNA encompasses chemically modified RNA nucleotides or DNA nucleotides. As commonly known RNA comprises the nucleotide U while DNA comprises the nucleotide T.

In accordance with items (d) and (a) of the above preferred embodiment the inhibitor may also be an expression vector or host, respectively being capable of producing an nucleic acid sequence as defined in any one of items (a) to (c).

An expression vector may be a plasmid that Is used to Introduce a specific transcript into a target cell. Once the expression vector Is inside the cell, the protein that Is encoded by the gene Is produced by the cellular-transcription and translation machinery ribosomal complexes. The plasmid Is in general engineered to contain regulatory sequences that act as enhancer and/or promoter regions and lead to efficient transcription of the transcript.

Non-limiting examples of expression vectors include prokaryotic plasmid vectors, such as the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), pEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Intvitrogen). For the formulation of a pharmaceutical composition a suitable vector is selected in accordance with good manufacturing practice. Such vectors are known in the art, for example, from Ausubel et al, Hum Gene Ther. 2011 April; 22(4):489-97 or Alay et al., Hum Gene Ther. May 2011; 22(5): 595-804.

A typical mammalian expression vector contains the promoter element, which mediates the initiation of transcription of mRNA, the protein coding sequence, and signals required for the termination of transcription and polyadenylation of the transcript. Moreover, elements such as origin of replication, drug resistance gene, regulators (as part of an inducible promoter) may also be included. The lac promoter Is a typical inducible promoter, useful for prokaryotic cells, which can be induced using the lactose analogue isopropylthiol-b-D-galactoside (“IPTG”). For recombinant expression and secretion, the polynucleotide of interest may be ligated between e.g. the PelB leader signal, which directs the recombinant protein in the periplasm and the gene III in a phagemid called pHEN4 (described in Ghahroudi et al, 1997, FEBS Letters 414:521-526). Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Alternatively, the recombinant (poly)peptide can be expressed in stable cell lines that contain the gene construct integrated into a chromosome. The co-transfection with a selectable marker such as dhfr, gpt, neomycin, hygromycin allows the identification and isolation of the transfected cells. The transfected nucleic acid can also be amplified to express large amounts of the encoded (poly)peptide. The DHFR (dihydrofolate reductase) marker is useful to develop cell lines that carry several hundred or even several thousand copies of the gene of interest. Another useful selection marker Is the enzyme glutamine synthase (GS) (Murphy et al. 1991, Biochem J. 227:277-279; Bebbington et al. 1992, Bio/Technology 10′169-175). Using these markers, the mammalian cells are grown in selective medium and the cells with the highest resistance are selected. As indicated above, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. For vector modification techniques, see Sambrook and Russel (2001), Molecular Cloning: A Laboratory Manual, 3 Vol. Generally, vectors can contain one or more origins of replication (on) and inheritance systems for cloning or expression, one or more markers for selection in the host, e.g., antibiotic resistance, and one or more expression cassettes. Suitable origins of replication (or) include, for example, the Col E1, the SV40 viral and the M 13 origins of replication.

The sequences to be inserted into the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of the transcription (e.g., translation initiation codon, promoters, enhancers, and/or insulators), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers, and/or naturally-associated or heterologous promoter regions. Preferably, the nucleotide sequence as defined in item (a) of the above preferred embodiment of the invention is operatively linked to such expression control sequences a flowing expression in prokaryotic or eukaryotic cells.

The host may be a prokaryotic or eukaryotic cell. A suitable eukaryotic host may be a mammalian cell, an amphibian cell, a fish cell, an insect cell, a fungal cell or a plant cell. Representative examples of bacterial cells are E. coli, Streptomyces and Salmonella typhimurium cells; of fungal cells are yeast cells; and of insect cells are Drosophila S2 and Spodoptera Sf9 cells. It is preferred that the cell is a mammalian cell such as a human cell. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells. The cell may be a part of a cell line, preferably a human cell line or a CHO cell line. Appropriate culture mediums and conditions for the above-described host cells are known in the art. The host is preferably a host cell and more preferably an isolated host cell. The host is also preferably a non-human host.

In accordance with a preferred embodiment of the first and second aspect of the invention the Immunotherapy comprises application of an immune checkpoint Inhibitor, preferably an inhibitor of ErbB2, EGFR, CD20, PD-1, PDL-1, CTLA4, IDO1, LAG3. TIM3. TIM-4, CXCL9, CXCL13, TIGIT, BTLA, CD137, OX40, VISTA, B7-H7, CD27, GITR, TGF-ß Signaling pathway, IL-15, PD-1 or PD-1L, preferably of PD-1 and/or PD-1L.

The mRNA expression level or the protein level of one or more selected from ErbB2, EGFR, CD20, CTLA4, IDO1, LAG3, TIM3, TIM-4, CXCL9, CXCL13, TIGIT, BTLA, CD137, OX40, VISTA, B7-H7, CD27, GITR, TGF-8 Signaling pathway, IL-15, PD-1 and PD-1L are known from the prior art to be involved in immune checkpoints. Accordingly, the mRNA of or the proteins ErbB2, EGFR, CD20. CTLA4, IDO1, LAG3, TIM3, TIM-4, CXCL9, CXCL13, TIGIT, BTLA, CD137, OX40, VISTA, B7-H7, CD27, GITR, TGF-ß Signaling pathway, IL-15, PD-1 and PD-1L are targets of immune checkpoint inhibitors. Particular preferred examples of such immune checkpoint inhibitors will be provided in the following.

In accordance with a preferred more embodiment of the first and second aspect of the invention the immune checkpoint Inhibitor Is selected from the group consisting of Trastuzumab, Cetuximab, Rituximab, Nivolumab, Pembrolizumab, Cemiplimab, Atezolizumab, Durvalumab, Avelumab, Ipilimumab, Relatlimab, LY3321367, MBF453, TSR-022, Urelumab, PFZ-05082566, 1-7F9 (IPH2101), GSK2831781, MED116469, MED116383, MOXR0916, Varlilumab, TRX518, NKG2D ligand-antitumour Fv fusion (preclinical development). Galunisertib. ALT-803 (IL-15-IL-15alpha-Sushi-Fc fusion complex) epacadostat, IMP321, and JNJ-83723283.

Trastuzumab is a therapeutic antibody binding to the HER2 receptor and thereby slowing down cell duplication.

Cetuximab is antibody against the epidermal growth factor receptor (EGFR) and used for the treatment of cancer, such as metastatic colorectal cancer, metastatic non-small cell lung cancer and head and neck cancer.

Rituximab is a chimeric monoclonal antibody against the protein CD20. It is used for the treatment of autoimmune disease and cancer.

Nivolumab (marketed as Opdivo) is an anti-PD-1 monoclonal antibody and is used to treat cancer. Pembrolizumab (formerly MK-3475 and lambrolizumab, trade name Keytruda) and Cemiplimab are further anti-PD-1 antibodies and used to treat cancer.

Atezolizumab is an antibody against the protein programmed cell death-ligand 1 (PD-L1) and used for cancer immunotherapy. Durvalumab and Avelumab are further antibodies against PD-L1 being useful for the treatment of cancer.

Ipilimumab is a monoclonal antibody against CTLA-4. It is used for the treatment of cancer, inter alia of melanoma, non-small cell lung carcinoma (NSCLC), small cell lung cancer (SCLC), bladder cancer and metastatic hormone-refractory prostate cancer.

Relatlimab (BMS-988016) is an anti-LAG3 antibody designed for the treatment of melanoma.

LY3321367, MBF453 and TSR-022 are anti-HAVCR2 monoclonal antibodies and useful for the treatment of cancer.

Urelumab (BMS-683513 or anti-4-1BB antibody) and Utomimulab (PF-05082588) are anti-CD137 antibodies. In more detail, they specifically bind to and activate CD137-expressing immune cells, thereby stimulating an immune response, in particular a cytotoxic T cell response against tumor cells.

IPH2101 is an anti-KIR (1-7F9) human monoclonal antibody developed for the treatment of patients with acute myeloid leukemia.

GSK2831781 is an anti-Lag3 antibody and used for the treatment of autoimmune diseases.

MED116489 is an anti-OX40 antibody being used for immunotherapy.

MED116383 is a human OX40 fusion protein and is also used in immunotherapy.

MOXR0918 is an anti-Ox40 antibody and is used for the treatment of solid tumors.

Varlilumab specifically binds CD27. It is used in the treatment of cancer, e.g. advanced breast or ovarian cancer.

TRX518 is an antibody blocking the interaction of glucocorticoid-induced TNF-superfamily receptor (GITR). The antibody is useful for the treatment of tumors.

Galunisertib is a small molecular inhibitor of TGF-beta and is used as cancer drug.

ALT-803 (IL-15-IL-15alpha-Sushi-Fc fusion complex) is an IL-15 superagonist complex that includes an IL-15 mutant (IL-15N72D) fused to an IL-15 receptor α/IgG1 Fc fusion protein. ALT-803 can trigger antigen-specific antitumor responses.

Epacadostat is a small molecule Inhibitor of indoleamine 2,3-dioxygenase-1) (IDO1) and is used in the treatment of cancer.

IMP321 (Eftilagimod alpha) Is a soluble version of LAG3 and is used to increase an immune response to tumors.

JNJ-83723283 is a monoclonal antibody directed against the negative immunoregulatory human cell surface receptor programmed cell death 1 protein (PD-1, PCDC-1), with potential immune checkpoint inhibitory and antineoplastic activity. Upon administration, anti-PD-1 monoclonal antibody JNJ-63723283 binds to PD-1, and inhibits the interaction with its ligands, programmed cell death 1 ligand 1 (PD-L1, PD-1L1) and PD-1 ligand 2 (PD-L2, PD-1L2). The inhibition of ligand binding prevents PD-1-mediated signaling and results in both T-cell activation and the induction of T-cell-mediated immune responses against tumor cells.

In accordance with another more preferred embodiment of the first and second aspect of the invention the anti-hormonal therapy comprises an anti-estrogen therapy and/or anti-progesterone and/or anti androgen therapy.

Estrogen (or oestrogen) is the primary female sex hormone. It is normally responsible for the development and regulation of the female reproductive system and secondary sex characteristics. Progesterone (P4) is an endogenous steroid being involved in the menstrual cycle, pregnancy, and embryogenesis of humans and other species. Androgen is the primary male sex hormone. It is normally responsible for the development and regulation of the male reproductive system and secondary sex characteristics. Estrogen, progesterone and androgen are both hormones being involved in tumorigenesis. In particular, estrogen-, androgen- or progesterone receptor-positive cancers are treated with drugs which suppress production or interfere with the action of these hormones in the body.

In accordance with a further preferred more embodiment of the first and second aspect of the invention the tumor is a cancer, preferably a carcinoma and is most preferably bladder cancer.

In the examples herein below expression levels of the HLA-G, L, H and J genes or proteins were determined in samples from bladder cancer patients.

In the case of bladder cancer or neoplastic lesions thereof it is preferred that the use comprises an instillation therapy into the bladder. The administration as instillation therapy is regarded as part of the invention particularly for the combination of immunological, chemotherapeutic, anti-hormonal, or anti-tyrosin kinase compounds together with anti-HLA agents as described as part of this application.

The present invention relates in a third aspect to a method for preparing a kit for predicting whether a subject having a tumor responds to a tumor treatment selected from (i) an immunotherapy, (ii) a chemotherapy, (iii) an anti-hormonal therapy, and (iv) an anti-tyrosin kinase therapy wherein the method comprises combining means for the detection of the level(s) of at least one nucleic acid molecule as defined herein above and/or at least one protein or peptide as defined herein above, and instructions how to use the kit.

The kit to be prepared implements a/the means required for conducting the invention of the invention in the format of a kit. For this reason the definitions and preferred embodiments provided herein above in connection with the first aspect of the invention are equally applicable to the kit of the invention.

A/the means for the detection and/or quantification of the nucleic acid molecule as exemplified as part of this invention may be one or more of the primer and probes as shown herein below in Table 1. However, any detection module being capable of quantifying nucleic acids such as arrays, NGS or other molecular systems would be appropriate as part of this invention. A/the means for the detection of the protein or peptide are preferably an antibody and/or protein binder and/or peptide binder (?) as described herein above. For detection and/or quantification the antibody and/or protein binder and/or peptide binder (?) may be labeled, e.g. by a fluorescent dye or a radiolabel. Examples of fluorescent dyes and radiolabels are also described herein above.

The various components of the kit may be packaged into one or more containers such as one or more vials. The vials may, in addition to the components, comprise preservatives or buffers for storage. The kit may comprise instructions how to use the kit, which preferably inform how to use the components of the kit for predicting whether a subject having a tumor responds to a tumor therapy as defined herein.

In a preferred embodiment of the third aspect of the invention the means comprise primer pairs and optionally a hydrolysis probe or other labelled primer or probe detection approaches for target sequence quantitation known to persons skilled in the art such as scorpion primers, FRET-probes or molecular beacons used for the sequence-specific detection of at least one nucleic acid molecule as defined herein above.

The primer pairs and optionally a hydrolysis probe are generally used for the specific detection of at least one nucleic acid molecule as defined herein above in a real time quantitative PCR a described herein above. Preferred primer pairs and hydrolysis probes are shown herein below in Table 1.

The hydrolysis probe designates the above-discussed sequence-specific DNA probe consisting of an oligonucleotide that is labelled with a fluorescent reporter which permits detection only after hybridization of the probe with its complementary sequence (e.g. a TaqMan probe). In more detail, hydrolysis probes are dual-labelled oligonucleotides. The 5′ end of the oligonucleotide is labelled with a fluorescent reporter molecule while the 3′ end is labelled with a quencher molecule. The sequence of the probe is specific for a region of interest in the amplified target molecule. The hydrolysis probe is designed so that the length of the sequence places the 5′ fluorophore and the 3′ quencher in close enough proximity so as to suppress fluorescence. During the extension phase of the PCR cycle the DNA polymerase synthesizes the complementary strand downstream of the PCR primers. When extension reaches the bound hydrolysis probe the 5′-3′ exonuclease activity of the DNA polymerase degrades the hydrolysis probe. Cleavage of the hydrolysis probe separates the fluorescent reporter molecule from the rest of the probe allowing the reporter molecule to fluoresce.

As regards the embodiments characterized in this specification, in particular in the claims, it Is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G: A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The Figures show.

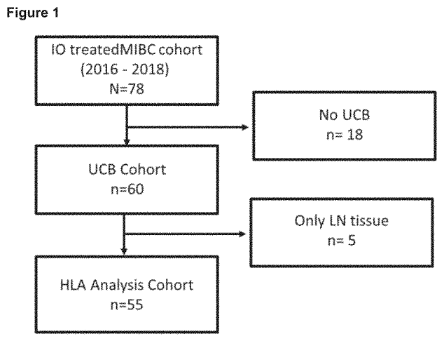

FIG. 1. Consort Diagram of advanced or metastatic urothelial cancer cohort. After exclusion of formalin-fixed paraffin-embedded (FFPE) blocks with insufficient and/or lymphnode tissues, tissues of 55 patients were available for analysis.

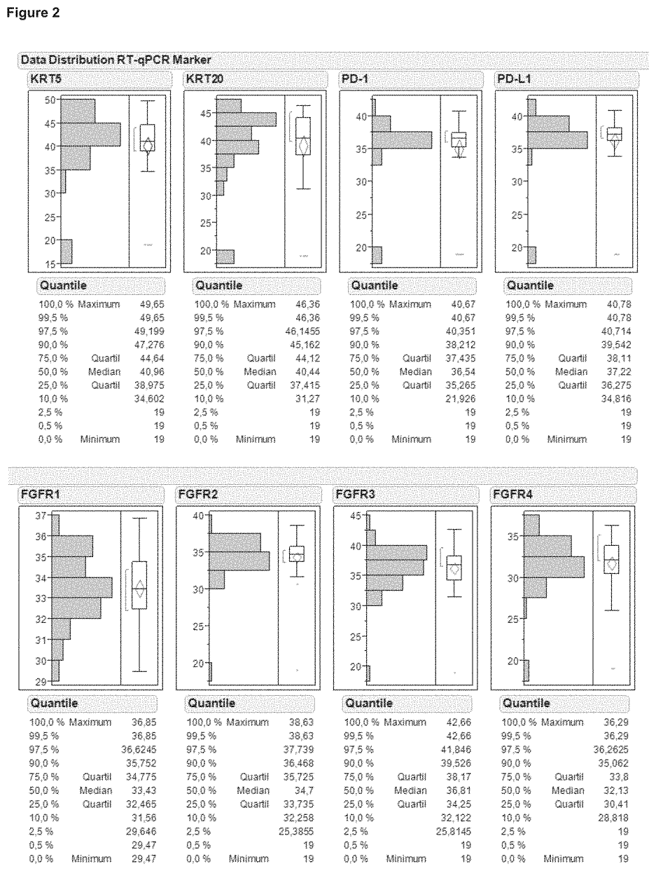

FIG. 2. Data distribution of luminal and basal subtype markers, check point target genes and FGFR1 to 4 gene expression as determined by RT-qPCR from tissues from muscle invasive bladder cancer patients.

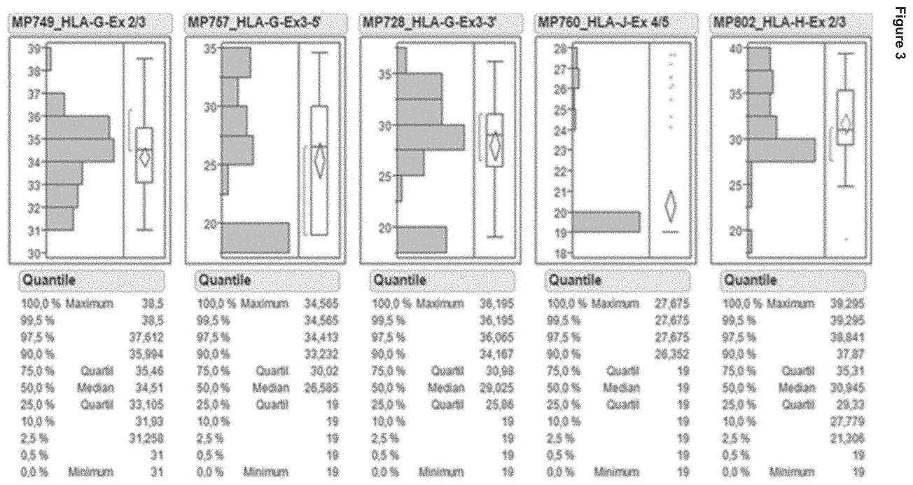

FIG. 3. Quantification of HLA-G, -H, -J, -L, -V mRNA expression by RT-qPCR assay of distinct exon regions. Relative mRNA expression is determined by the 40-DCT method using CALM2 as reference gene. The higher the 40-DCT value, the higher the gene expression.

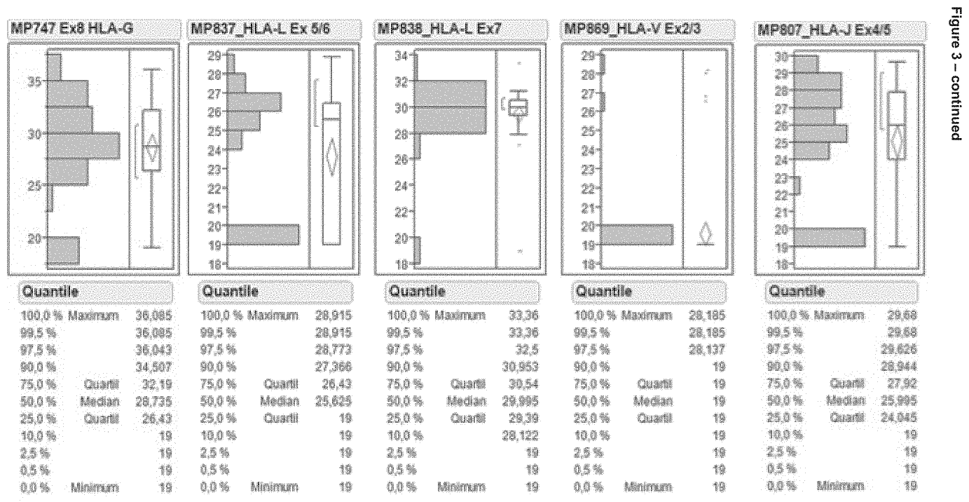

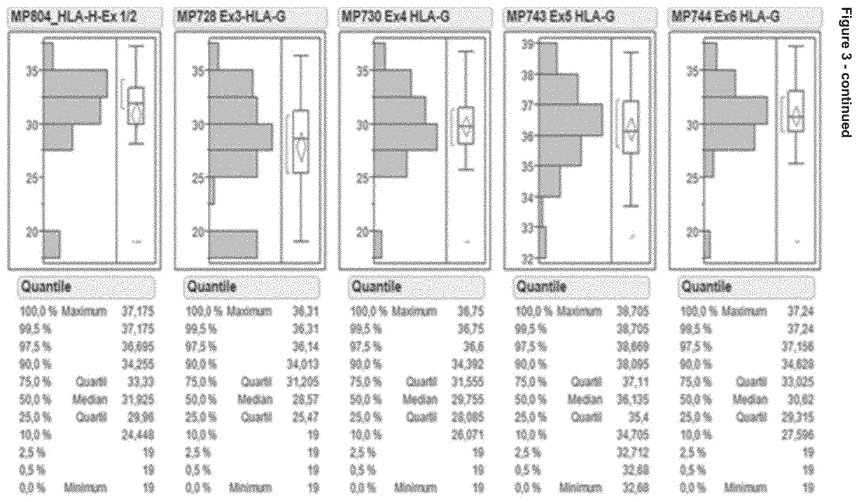

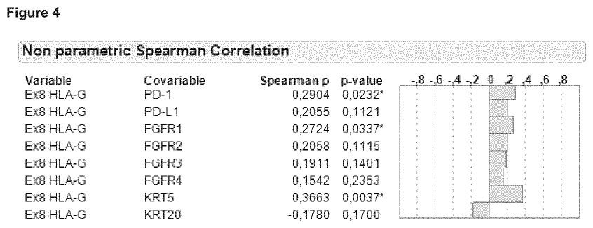

FIG. 4. Intergene spearman correlation of luminal and basal subtype markers, check point target genes. FGFR1 to 4 genes and exon 8 mRNA expression analysis of HLA-G, as determined by RT-qPCR from tissues from muscle invasive bladder cancer patients.

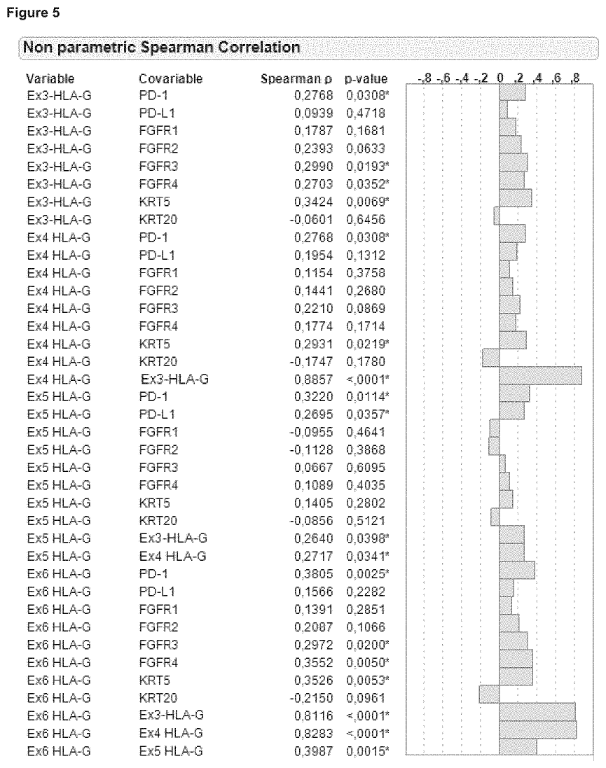

FIG. 5. Intergene spearman correlation of luminal and basal subtype markers, check point target genes, FGFR1 to 4 genes and HLA-G exon 3 to 6 mRNA expression analysis as determined by RT-qPCR in tissues from muscle invasive bladder cancer patients (n=61).

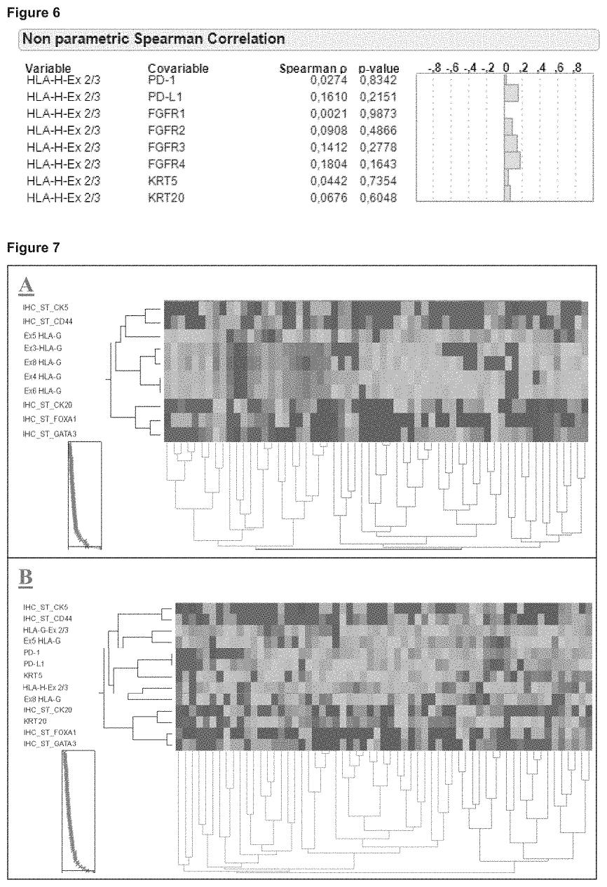

FIG. 6. Correlation of HLA-H mRNA expression in urothelial cancer patients with FGFR receptors, PD-1, PD-L1 and markers for basal and luminal cell type.

FIG. 7. Cluster analysis of HLA genes with immune histological and molecular assessed urothellal markers. Red highlights high gene expression, whereas blue depict low gene expression. Genes are depicted on the left side of the cluster analysis. Each column represents a cystectomy UBC sample form a patient

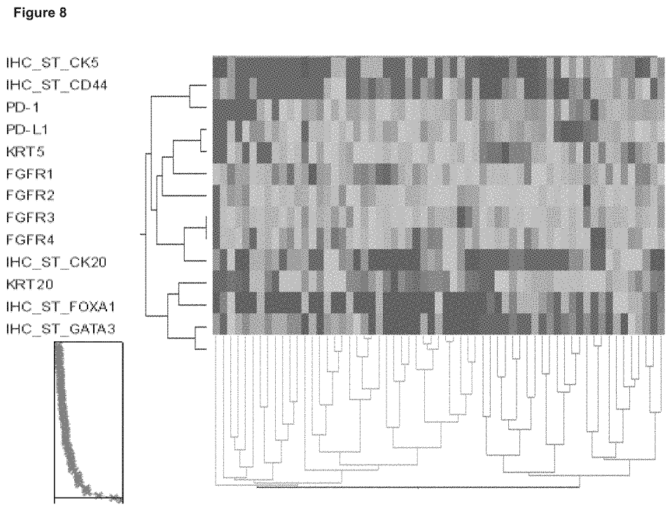

FIG. 8. Cluster analysis of FGF receptor genes with PD-1, PD-L1 and basal and luminal markers. Red highlights high gene expression, whereas blue depict low gene expression. Genes are depicted on the left side of the cluster analysis. Each column represents a cystectomy UBC sample form a patient

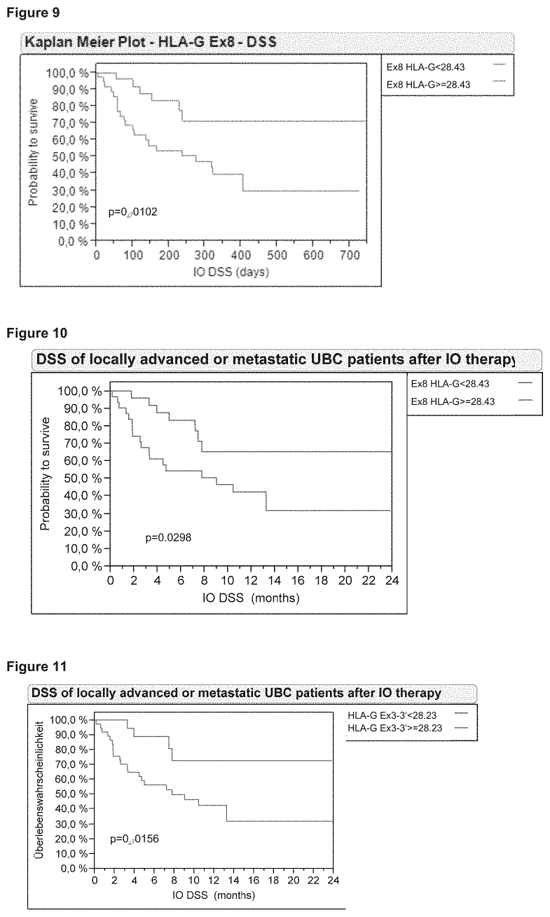

FIG. 9. Kaplan Meier Plot displaying disease specific survival (DSS) probability from muscle invasive bladder cancer patients based on stratification by HLA-G exon 8 expression as quantified by RT-qPCR assay. Relative mRNA expression is determined by the 40-DCT method using CALM2 as reference gene.

FIG. 10. Kaplan Meier Plot displaying disease specific survival (DSS) probability from muscle invasive bladder cancer patients having locally advanced or metastatic UBC (n=57) based on stratification by HLA-G exon 8 expression as quantified by RT-qPCR assay. Relative mRNA expression is determined by the 40-DCT method using CALM2 as reference gene.

FIG. 11. Kaplan Meier Plot displaying disease specific survival (DSS) probability from muscle invasive bladder cancer patients having locally advanced or metastatic UBC (n=57) based on stratification by HLA-G exon 3 expression as quantified by RT-qPCR assay. Relative mRNA expression is determined by the 40-DCT method using CALM2 as reference gene.

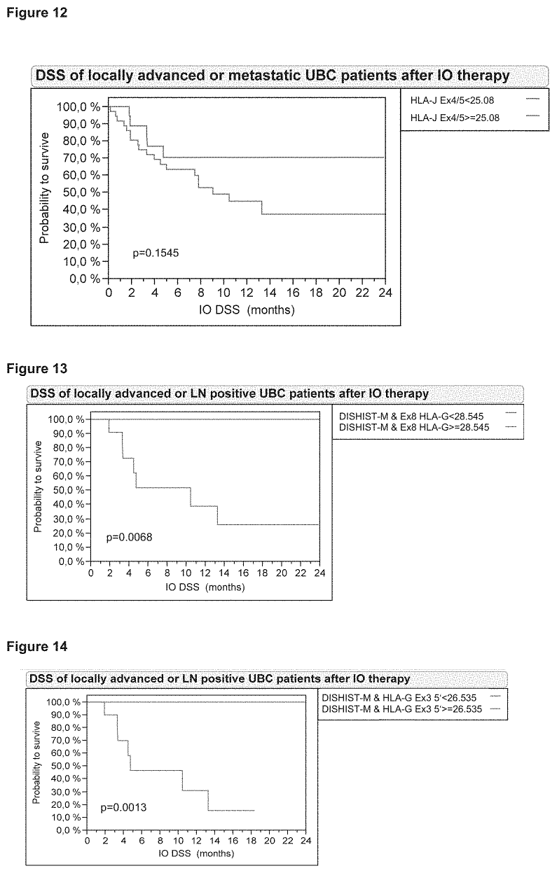

FIG. 12. Kaplan Meier Plot displaying disease specific survival (DSS) probability from muscle invasive bladder cancer patients having locally advanced or metastatic UBC (n=57) based on stratification by HLA-J Exon 4/5 expression as quantified by RT-qPCR assay. Relative mRNA expression is determined by the 40-DCT method using CALM2 as reference gene.

FIG. 13. Kaplan Meier Plot displaying disease specific survival (DSS) probability from muscle invasive bladder cancer patients having locally advanced or lymph node positive UBC (n=20) based on stratification by HLA-G exon 8 expression as quantified by RT-qPCR assay. Relative mRNA expression is determined by the 40-DCT method using CALM2 as reference gene.

FIG. 14. Kaplan Meier Plot displaying disease specific survival (DSS) probability from muscle invasive bladder cancer patients having locally advanced or lymph node positive UBC (n=20) based on stratification by HLA-G exon 3 expression as quantified by RT-qPCR assay. Relative mRNA expression is determined by the 40-DCT method using CALM2 as reference gene.

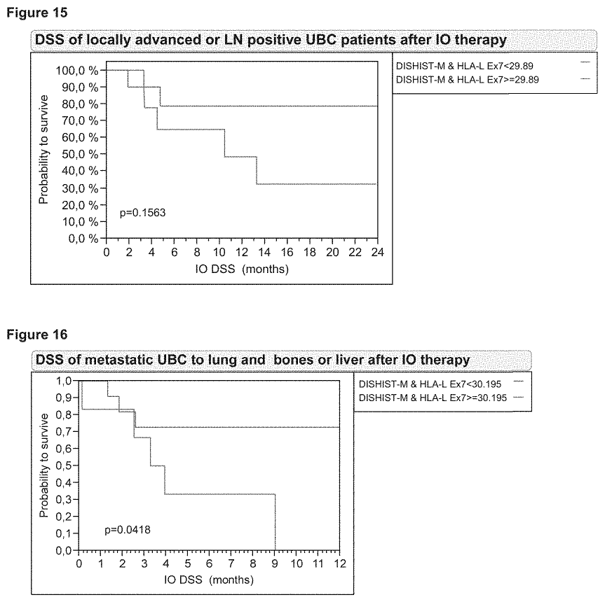

FIG. 15. Kaplan Meier Plot displaying disease specific survival (DSS) probability from muscle invasive bladder cancer patients having locally advanced or lymph node positive UBC (n=19) based on stratification by HLA-L exon 7 expression as quantified by RT-qPCR assay. Relative mRNA expression is determined by the 40-DCT method using CALM2 as reference gene.

FIG. 16. Kaplan Meier Plot displaying disease specific survival (DSS) probability from muscle invasive bladder cancer patients having metastasized to lung and bones or liver (n=17) based on stratification by HLA-L exon 7 expression as quantified by RT-qPCR assay. Relative mRNA expression is determined by the 40-DCT method using CALM2 as reference gene.

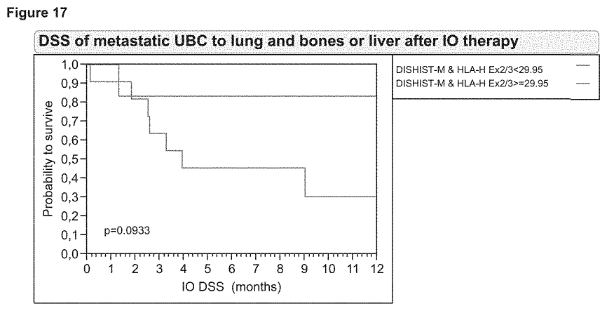

FIG. 17. Kaplan Meier Plot displaying disease specific survival (DSS) probability from muscle invasive bladder cancer patients having metastasized to lung and bones or liver (n=17) based on stratification by HLA-H exon 2/3 expression as quantified by RT-qPCR assay. Relative mRNA expression is determined by the 40-DCT method using CALM2 as reference gene.

The examples illustrate the invention.

### Example 1

### EXAMPLES

### Example 1: HLA Profiling in Advanced, Chemotherapy Refractory Urothelial Cancer

Transurethral resection (TUR) biopsies and cystectomy samples from primary tumors being refractory to chemotherapy and thereafter undergoing first or second line immuneoncology (“IO”) treatment by PD-1 and PD-L1 checkpoint inhibitor drugs (i.e. Atezolizumab, Nivolumab and Pembrolizumab) were analyzed for HLA expression and associated with histopathoiogical and molecular parameters as well as response to IO treatment and disease specific survival after IO.

Seventy-two newly diagnosed patients with histologically confirmed urothelial cancer, including bladder cancer and upper urothelial tract carcinoma were enrolled in the study between 2016 and 2018. Nivolumab. Pemprolizumab and Atezumab were given as 1st, 2nd and 3rd line mono-treatment according to approved Instructions. All hematoxylin-eosin (HE) stained tumor tissue sections from samples of the cohort were evaluated and classified according to TNM-classification (2017) of the UICC by two uro-pathologists. Rare histological variants were classified according to the World Health Organization (WHO 2016) classification of genitourinary tumors. After central histopathological review 18 tissues were excluded for not having sufficient tumor material or not being urothelial cancer. From patients only lymphnode tissue was available and therefore excluded from primary analysis of prognostic and/or predictive effects of HLA gene expression (see FIG. 1: Consort Diagram). Data base closure for clinical data was done on Oct. 16, 2018 in conjunction with a parallel FDA submission.

For mRNA detection, RNA was extracted from FFPE tissue from TUR biopsies, cystectomy and corresponding mapping bladder tissue using commercial kits (Xtract, Stratifyer). For each reaction, 2.5 μl total RNA extracted from FFPE sections were mixed with 2.5 μl assay-mix, 2.5 μl enzyme-mix and 2.5 μl water in one well of a 96-well-optical reaction plate. Measurements of the PCR reaction were done according to the instructions of the manufacturer with a Versant kPCR Cycler (Siemens) or a Light Cycler 480 (Roche) under appropriate conditions (5 min 50° C., 1 cycle; 20 s 95° C., 1 cycle; 15 s 95° C.; 1 min 60° C., 40 cycles). The relative mRNA expression was associated with response to 10 treatment determined based on RECIST (Response Evaluation Criteria in Solid Tumors) criteria as assessed at the individual sites and with disease specific survival as determined from start of IO treatment to cancer specific death. Partition testing using biostatistical JMP SAS 9.0.0 (SAS, Cary. N.C., USA) were performed to evaluate the possible differences in response to IO treatment.

For a detailed analysis of gene expression by RT-qPCR methods, primers flanking the region of interest and a fluorescently labeled probe hybridizing in-between were utilized. Target-specific primers and probes were selected using the NCBI primer designing tool (www.ncbi.nlm.nih.go). RNA-specific primer/probe sequences were used to enable RNA-specific measurements by locating primer/probe sequences across exon/exon boundaries. Furthermore, primers/probes were selected not to bind to sequence regions with known polymorphisms (SNPs). In case multiple isoforms of the same gene existed, primers were selected to amplify all relevant or selected splice variants as appropriate. All primer pairs were checked for specificity by conventional PCR reactions. After further optimization of the primers/probes, the primers and probes listed in Table 1 gave the best results. These primers/probes are superior to primers/probes known from the prior art. e.g., in terms of specificity and amplification efficiency. To standardize the amount of sample RNA, the CALM2 was selected as reference gene, since they were not differentially regulated in the samples analyzed. TaqMan validation experiments were performed showing that the efficiencies of the target and the control amplifications were approximately equal, which is a prerequisite for the relative quantification of gene expression by the comparative ACT method.

TABLE 1
Used primers and probes for HLA mRNA quantitation

| Gen | For_Primer | Probe | Rev-Primer |
|---|---|---|---|
| HLA-G- | GGCCGGAGTATTGGGAAGA | CAAAGGCCCACGCACAGACTGACA | GCAGGGTCTGCAGGTTCATT |
| Ex3 | | | |
| HLA-G | CTGCGGCTCAGATCTCCAA | CGCAAGTGTGAGGCGGCCAAT | CAGGTAGGCTCTCCTTTGTTCAG |
| Ex4 | | | |
| HLA-G | CACCACCCTGTCTTTGACTATGAG | ACCCTGAGGTGCTGGGCCCTG | AGTATGATCTCCGCAGGGTAGAAG |
| Ex5 | | | |
| HLA-G | CATCCCCATCATGGGTATCG | TGCTGGCCTGGTTGTCCTTGCA | CCGCAGCTCCAGTGACTACA |
| Ex6 | | | |
| HLA-G | GACCCTCTTCCTCATGCTGAAC | CATTCCTTCCCCAATCACCTTTCCTGTT | CATCCCAGCCCCTTTTCTG |
| Ex8 | | | |
| HLA-G | TTCATCGCCATGGGCTACG | CGACACGCAGTTCGTGCGGTTC | ATCCTCGGACACGCCGAGT |
| Ex3-5 | | | |
| HLA-G | CCGAACCCTCTTCCTGCTGC | CGAGACCTGGGCGGGCTCCC | GCGCTGAAATACCTCATGGA |
| Ex2/3 | | | |
| HLA-H | GAGAGAACCTGCGGATCGC | AGCGAGGGCGGTTCTCACACCATG | CCACGTCGCAGCCATACAT |
| Ex2/3 | | | |
| HLA-H | GAGAGAACCTGCGGATCGC | ACCAGAGCGAGGGCGGTTCTCACAC | CGGGCCGGGACATGGT |
| KRT5 | CGCCACTTACCGCAAGCT | TGGAGGGCGAGGAATGCAGACTCA | ACAGAGATGTTGACTGGTCCAACTC |
| KRT20 | GCGACTACAGTGCATATTACAGAC | TTGAAGAGCTGCGAAGTCAGATTAAGGATGCT | CACACCGAGCATTTTGCAGTT |
| | AA | | |
| PD-1 | GGCCAGCCCCTGAAGGA | ACCCCTCAGCCGTGCCTGTGTTC | GGAAATCCAGCTCCCCATAGTC |
| PDL1 | TGGCATCCAAGATACAAACTCAA | CAAAGTGATACACATTTGGGAGGAGACGTAA | TTGAAGATCAGAAGTTCCAATGCT |
| CALM2 | GAGCGAGCTGAGTGGTTGTG | TCGCGTCTCGGAAACCGGTAGC | AGTCAGTTGGTCAGCCATGCT |
| HLA-L | CCTGCTCCGCTATTACAACCA | CGAGGCCGGTATGAACAGTTCGCCTA | CGTTCAGGGCGATGTAATCC |
| Ex2/3 | | | |
| HLA-L | GCTGTGGTGCTGCTGCG | AGAAAAGCTCAGGCAGCAATTGTGCTCAG | CATAGTCCTCTTTACAAGTATCATGAGA |
| Ex5/6 | | | TG |
| HLA-L | TCCTCTCTGCTCAGCTCTCCTA | CTCTCCCTTCCCTGAGTTGTAGTAATCCTAGCA | GCTTTATAGATCCATGAGTTTGCATTA |
| Ex7 | | CT | |
| HLA-J | CAAGGGGCTGCCCAAGC | CATCCTGAGATGGGTCACACATTTCTGGAA | CCTCCTAGTCTTGGAACCTTGAGAAGT |
| Ex4/5 | | | |

The determination of luminal and basal subtypes in the UC cohort by RT-qPCR revealed a broad dynamic range of KRT5 and KRT20 mRNA ranging from 40-DCT values of 19 to 48 in similar ranges. The mRNA expression of PD-1 and PD-L1 ranged from 19 to 41. The dynamic range for the FGFR genes differed markedly within the FGFR family. The relative FGFR1 mRNA ranged from 29 to 37, FGFR2 mRNA from 19 to 39, FGFR3 mRNA from 19 to 43 and FGFR4 mRNA from 19 to 38 (FIG. 2).

In addition to the mRNA expression analysis of luminal and basal markers, PD-1, PD-L1 and the FGFR family, the expression profile of classical HLAs as well as exon expression of HLA genes and pseudogenes have been carried out (FIG. 3).

Non-parametric spearman correlation of the FGFR genes 1-4, PD-1, PD-L1, basal and luminal markers as well as exon 8 HLA primer sets reveals a strong and significant correlation of PD-1 (spearman rho 0.2904, p=0.0232) in urothelial tumors expressing HLA-G exon 8. Besides PD-1, high FGFR1 (spearman rho 0.2724, p=0.0337) expression is also associated with HLA-G exon 8 expression. However, no significant correlation could be observed with any HLA for the luminal like urothelial carcinomas (FIG. 4).

Surprisingly, spearman correlation of luminal and basal subtype markers, check point target genes. FGFR1 to 4 genes with remaining HLA-G exons reveals a strong and significant association of HLA-G with the check point marker PD-1 as indicated by an similar high coexpression with all HLA-G exons (PD-1 in exon 3 3′end: spearman rho 0.2768, p=0.0308; PD-1 in exon 4: spearman rho 0.2768, p=0.0308; PD-1 in exon 5: spearman rho 0.3220, p=0.0114; PD-1 in exon 6: spearman rho 0.3805, p=0.0025)(FIG. 5). These interesting finding could only be confirmed in HLA-G exon 5 for PD-L1 (spearman rho 0.2695, p=0.0357). For exon 3 3′end, high significant correlation can also be observed for FGF receptor 3 (spearman rho 0.2990, p=0.0193) and 4 (spearman rho 0.2703, p=0.0352). This association could not be determined for exon 4, though exon 4 expression was associated with high mRNA expression of the basal cell marker KRT5 (spearman rho 0.2931, p=0.0219). The basal marker KRT5 (spearman rho 0.3526, p=0.0053) showed also significant correlation with HLA-G exon 6. In addition, FGF receptors 3 (spearman rho 0.2972, p=0.0200) and 4 (spearman rho 0.3552, p=0.0050) show also significant correlations with HLA-G exon 6 In mRNA expression.

In addition, non parametric Spearman correlation analysis of luminal and basal subtype markers, PD1, PD-L1 and FGFR1 to 4 genes has also been done for HLA-H (FIG. 6). However, no correlation between HLA-H expression and luminal or basal markers or check point inhibitors could be observed. Further, cluster analysis of FGF receptor genes with PD-1, PD-L1 and basal and luminal markers was carried out. The analysis revealed that PD-1 and PD-L1 expression occurs in rather basal urothelial cancer subtypes. In addition, FGFR1 mRNA is also higher expressed in Cytokeratin 5 positive tumors, whereas FGF receptors 2 to 4 show higher expression in rather luminal urothelial cancer subtype.

Cluster analysis of HLA genes with immune histological assessed urothelial cancer markers, reveal that HLA-G expression mainly occurs in basal urothelial cancer subtypes (IHC_ST_CK5). The basal urothelial cancer subtype can further be divided by HLA mRNA expression. Some basal tumor subtypes show a high HLA-G expression (FIG. 7 A). Finally, cluster analysis of HLA Exon 8 expression with immune histological cell and subclassification markers (CK5, CD44, CK20, FOXA1, GATA3) PD-1, PD-L1 as well as HLA-H expression was performed. The analysis revealed that HLA-G Exon 5 and Exon 8 expression and HLA-H expression as well as PD-1. PD-L1 can rather be assigned to the basal subtype. However. HLA-G. HLA-H and PD-1 and PD-L1 expression could also be observed in luminal urothelial tumors (FIG. 7 B). In addition. In silico promoter analysis revealed several estrogene and response elements (ERE) as well as a progesterone response element (PRE) in the HLA-G gene. This Indicates the important potential of HLA-G expression not only in basal but also in luminal cancer subtypes. Since mRNA exon and exon/exon junction expression varies within the luminal and basal cancer subtypes single exon expression and exon/exon Junction analysis as a stratification tool should be applied in basal as well as luminal urothelial cancer subtypes. Surprisingly, in silico analysis of the HLA-H promoter region also revealed several estrogen response elements. Together with the cluster analysis, this underlines the important role of the pseudogene HLA-H as a further stratification tool in urothelial cancer. As depicted in FIG. 8, further cluster analysis of FGF receptor genes has been carried out with PD-1, PD-L1 and basal and luminal markers. The analysis proved, that PD-1 and PD-L1 expression occurs in rather basal urothelial cancer subtypes. In addition, FGFR1 mRNA is also higher expressed in Cytokeratin 5 positive tumors, whereas FGF receptors 2 to 4 show higher expression in rather luminal urothelial cancer subtype. This demonstrates the representatively of the cohort analyzed for HLA gene Interactions.

### Example 2: Exon Expression of Different HLA Genes in Urothelial Cancer as Marker for Disease Specific Survival (DSS)

To determine the predictive value of HLA gene expression in bladder cancer tissues of advanced or metastatic urothelial cancer patients undergoing immune-oncological checkpoint therapy (IO therapy) (i.e. Atezolizumab, Nivolumab or Pembrolizumab) were assessed based on detailed clinical follow up data, which comprised i.a. WHO grading, primary metastatic sites, start of IO treatment, time point of cancer specific death or last contact date. The immune-oncological disease specific survival was calculated from start of IO therapy to cancer specific death or last contact and censored respectively.

As depicted in FIG. 9 relevance of changes of HLA-G mRNA expression on disease specific survival (DSS) of urothelial cancer patients was analyzed. When taking all available tissues including metastatic lymphnodes into account (n=60) Kaplan Meier analysis revealed that an increased HLA-G Exon 8 mRNA expression above 28.43 40-DCT values indicated worse disease specific survival (p=0.0102).

However, to exclude non cancer associated effects of HLA expression by non-tumor-associated lymphocytes in the lymph nodes, the metastatic lymph node tissues were excluded from the subsequent analysis, leaving 57 samples for survival analysis as depicted in FIG. 1. As depicted in FIG. 10 high HLA-G Exon 8 mRNA expression (>=28.43) was significantly associated with inferior disease specific survival with HLA-G Exon 8 positive patients having a survival probability of 35% after 2 years, while HLA-G Exon 8 negative patients had a survival probability of 65% after 2 years (p=0.0298).

As the examined HLA-G specific Exon 8 region is not translated into protein further confirmatory analysis has been performed by determining the Exon 3 region of HLA-G, which is part or the translated region close to the signal peptide of HLA-G. As depicted in FIG. 11 high HLA-G Exon 3 mRNA expression (>=28.23) was significantly associated with inferior disease specific survival with HLA-G Exon 3 positive patients having a survival probability of 30% after 2 years, while HLA-G Exon 8 negative patients had a survival probability of 70% after 2 years (p=0.0158).

Next the prognostic value of other HLA genes in the total cohort was analyzed. Special focus has been on currently classified “pseudogenes” as exemplified for HLA-J, H, V or L. As depicted in FIG. 12 high HLA-J Exon 4/5 mRNA expression (>=25.08) was associated with inferior disease specific survival with 36 HLA-J Exon 4/5 positive patients having a survival probability of 35% after 2 years, while 19 HLA-J Exon 4/5 negative patients had a survival probability of 70% after 2 years.

To further elucidate the relevance of HLA-G expression on survival after IO therapy the analysis was further specified by analyzing only primary tumor tissue and in addition also taking the primary metastatic site into account. This is based on initial findings that IO therapy has differential effects depending on the site of metastasis with e.g. visceral metastasis into the liver being less effective, probably due to the fact that PD1 positive T-cells are being excluded from the liver in metastatic urothelial cancer patients independent of classical checkpoint mechanisms (Eckstein M, Sikic D, Strissel P L, Erimeier F. Evolution of PD-1 and PD-L1 Gene and Protein Expression in Primary Tumors and Corresponding Liver Metastases of Metastatic Bladder Cancer. Eur Urology 2018.). Therefore the patients were grouped according to the first manifestation of metastasis with local advancement, locoregional lymph nodes or extraregional retroperitoneal lymph nodes being categorized as 0 or 0.5, respectively, while dissemination into the bones, liver, lung, lung and bone or lung and liver were categorized with increasing indices (1, 2, 3, 4, 5; respectively). For this analysis 54 datasets from primary tumor tissues with sufficient clinical date and primary tumor tissue material were available, with 19 patients having local advancement or lymph node metastasis, while 17 patients had initially metastasized to bone or liver and 18 patients having metastasized with lung involvement either as singular site or in combination with bone or liver involvement, while all of them had been treated with IO drugs and predominantly >1^{st} line setting (74%).

In urothelial bladder cancer patients having advanced or lymph node positive disease high mRNA expression of HLA-G was associated with inferior disease specific survival determined from initiation of IO treatment to cancer specific death. As exemplified in FIG. 13 high HLA-G Exon 8 mRNA expression (>=28.545) had significant worse outcome with 11 HLA-G Exon 8 positive patients having a survival probability of only 25% after 2 years, while the 9 HLA-G Exon 8 negative patients had a survival probability of 100% after 2 years (p=0.0068).

As the examined HLA-G specific Exon 8 region is not translated into protein further confirmatory analysis has been performed by determining the Exon 3 region of HLA-G, which Is part of the translated region close to the signal peptide of HLA-G.

As depicted in FIG. 14 high HLA-G Exon 3 mRNA expression (>=28.535) was significantly associated with inferior disease specific survival with 10 HLA-G Exon 3 positive patients having a survival probability of only 15% after 2 years, while 10 HLA-G Exon 3 negative patients had a survival probability of 100% after 2 years (p=0.0013). This resembles the predictive value of HLA-G Exon 8 mRNA expression and further proves that HLA-G expression is associated with worse outcome despite treatment with check point inhibiting IO drugs in advanced and node positive disease situations.

Next it was examined whether other HLA genes, being classical or non-classical or being known genes or yet assigned to be pseudogenes, were predictive for IO outcome in urothelial bladder cancer.

As one example, assays were developed to quantify the mRNA of the “pseudogene” HLA-L at the similar region at the 3′end of the “pseudogene” analogous to the Exon 8 region of HLA-G. As depicted in FIG. 15 high HLA-L Exon 7 mRNA expression (>=29.89) was associated with inferior disease specific survival with 10 HLA-L Exon 7 positive patients having a survival probability of only 30% after 2 years, while 10 HLA-L Exon 7 negative patients had a survival probability of 80% after 2 years. However, this association did not reach statistical significance by log-rank test due to crossing of the survival curves. It can be argued, that on the one hand the sample size is still low, on the other hand the log-rank test might not be valid in this case, as a very early case after 1 month does have an exaggerated effect on the p-value and therefore might not be optimal to assess risk.

This Indicates that not only HLA-G, but also other HLA genes and/or pseudogenes are associated with worse outcome despite treatment with check point Inhibiting IO drugs. From a therapeutic standpoint this indicates, that not only HLA-G but simultaneously other HLA-genes and/or pseudogenes should be targeted to circumvent or break resistance towards IO drugs.

Next it was examined whether HLA genes are also predictive in most aggressive situations from tumorbiological standpoint, when multiple organs particularly including the lung have already been metastasized as determined by CT scan at diagnosis before IO therapy. As depicted in FIG. 16 high HLA-L Exon 7 mRNA expression (>=30.195) was associated with inferior disease specific survival with 16 HLA-L Exon 7 positive patients having a survival probability of only 0% after 1 year, while 11 HLA-L Exon 7 negative patients had a survival probability of 70% after 1 year (p=0.0418)

In this highly metastasized situation also other “pseudogenes” were significant as exemplified by HLA-H. As depicted in FIG. 17 high HLA-H Exon 2/3 mRNA expression (>=29.95) was associated with inferior disease specific survival with HLA-H Exon 2/3 mRNA positive patients having a survival probability of only 30% after 1 year, while HLA-H Exon 2/3 mRNA negative patients had a survival probability of 80% after 1 year.

1. A method for predicting whether a subject having a tumor responds to a tumor therapy selected from
(i) an immunotherapy,
(ii) a chemotherapy,
(iii) an anti-hormonal therapy, and
(iv) an anti-tyrosin kinase therapy,
wherein the method comprises
(A) determining the level(s) of at least one nucleic acid molecule and/or at least one protein or peptide in a sample obtained from said subject,
wherein the at least one nucleic acid molecule Is selected from nucleic acid molecules
(a) encoding a polypeptide comprising or consisting of the amino acid sequence of any one of SEQ ID NOs 2 and 4 to 8,
(b) consisting of the nucleotide sequence of any one of SEQ ID NOs 8 and 10 to 12,
(c) encoding a polypeptide which Is at least 85% identical, preferably at least 90% identical, and most preferred at least 95% identical to the amino acid sequence of (a),
(d) consisting of a nucleotide sequence which Is at least 95% identical, preferably at least 96% identical, and most preferred at least 98% identical to the nucleotide sequence of (b),
(e) consisting of a nucleotide sequence which is degenerate with respect to the nucleic acid molecule of (d),
(f) consisting of a fragment of the nucleic acid molecule of any one of (a) to (e), said fragment comprising at least 250 nucleotides, preferably at least 300 nucleotides, more preferably at least 450 nucleotides, and most preferably at least 600 nucleotides, and
(g) corresponding to the nucleic acid molecule of any one of (a) to (f), wherein T is replaced by U, and
wherein the at least one protein or peptide is selected from proteins or peptides being encoded by the nucleic acid molecule of any one of (a) to (g); and
(B) comparing the level(s) of (A) with the level(s) of the at least one nucleic acid molecule and/or the at least one protein or peptide in a sample obtained from one or more subjects that responded to one or more of the therapies of (i) to (ii) or a corresponding pre-determined standard,
wherein increased level(s) of (A) as compared to the level(s) or pre-determined standard of (B) indicate(s) that the subject will not respond to the tumor therapy and substantially the same or decreased level(s) of (A) as compared to the level(s) of (B) indicate(s) that the subject will respond to the tumor therapy; or
(B′) comparing the level(s) of (A) with the level(s) of the at least one nucleic acid molecule and/or the at least one protein or peptide in a sample obtained from one or more subjects that did not respond to one or more of the therapies of (i) to (iii) or a corresponding pre-determined standard,
wherein decreased level(s) of (A) as compared to the level(s) or pre-determined standard of (B) Indicate(s) that the subject will respond to the tumor therapy and substantially the same or increased level(s) of (A) as compared to the level(s) of (B′) indicate(s) that the subject will not respond to the tumor therapy.

2. The method of claim 1, wherein any one of SEQ ID NOs 2 and 4 to 6 is SEQ ID NO: 5 or 6, and any one of SEQ ID NOs 8 and 10 to 12 is SEQ ID NO: 11 or 12.

3. The method of claim 1 or 2, further comprising determining the mRNA expression level or the protein level of one or more selected from ErbB2, EGFR, CD20, CTLA4, IDO1, LAG3, TIM3, TIM-4, CXCL9, CXCL13, TIGIT, BTLA, CD137, OX40, VISTA, B7-H7, CD27, GITR, TGF-ß Signaling pathway, IL-15, PD-1 and PD-1L, preferably of PD-1 or PD-1L.

4. A binding molecule, preferably an inhibitor of at least one nucleic acid molecule as defined in claim 1 or 2 or at least one protein or peptide as defined in claim 1 or 2 for use in the treatment of a tumor in a subject, wherein the inhibitor Is to be used in combination with
(i) an immunotherapy;
(ii) a chemotherapy;
(iii) an anti-hormonal therapy; and/or
(iv) an anti-tyrosin kinase therapy.

5. The binding molecule, preferably the inhibitor for use of claim 4, wherein the subject has been predicted to not respond to
(i) an immunotherapy;
(ii) a chemotherapy;
(iii) an anti-hormonal therapy; and/or
(iv) an anti-tyrosin kinase therapy
by the method of any one of claims 1 to 3.

6. The inhibitor for use of claim 4 or 5, wherein the inhibitor is a small molecule Inhibitor, a nucleotide-based inhibitor or an amino acid-based inhibitor.

7. The Inhibitor for use of claim 6, wherein the nucleotide-based Inhibitor or amino acid-based inhibitor is an aptamer, a ribozyme, a siRNA, a shRNA, an antisense oligonucleotide, a CRISPR-endonuclease-based construct, a meganuclease, a zinc finger nuclease, or a transcription activator-like (TAL) effector (TALE) nuclease and the amino acid-based inhibitor is an antibody or a protein drug.

8. The inhibitor for use of claim 7, wherein the protein drug is an antibody mimetic, preferably selected from affibodies, adnectins, anticalins, DARPins, avimers, nanofitins, affilins, Kunitz domain peptides, Fynomers®, trispecific binding molecules and probodies.

9. The inhibitor for use of claim 6 or 7, wherein the nucleotide-based inhibitor comprises
(a) a nucleic acid sequence which comprises or consists of a nucleic acid sequence being complementary to at least 12 continuous nucleotides of a nucleic acid sequence selected from SEQ ID NOs 8 and 10 to 12 or a sequence being at least 80% identical thereto,
(b) a nucleic acid sequence which comprises or consists of a nucleic acid sequence which is at least 80% identical to the complementary strand of one or more nucleic acid sequences selected from SEQ ID NOs 8 and 10 to 12,
(c) a nucleic acid sequence which comprises or consists or a nucleic acid sequence according to (a) or (b), wherein the nucleic acid sequence is DNA or RNA,
(d) an expression vector expressing the nucleic acid sequence as defined in any one of (a) to (c), preferably under the control of a tumor-specific promoter, or
(e) a host comprising the expression vector of (d).

10. The method of any one of the preceding claims or the inhibitor for use of any one of the preceding claims, wherein the immunotherapy comprises application of an immune checkpoint inhibitor, preferably an inhibitor of ErbB2, EGFR, CD20, PD-1, PDL-1, CTLA4, IDO1, LAG3, TIM3, TIM-4, CXCL9, CXCL13, TIGIT, BTLA, CD137, OX40, VISTA, B7-H7, CD27, GITR, TGF-9 Signaling pathway, IL-15, PD-1 or PD-1L, preferably of PD-1 and/or PD-1L.

11. The method of claim 10 or the inhibitor for use of claim 10, wherein the immune checkpoint Inhibitor is selected from the group consisting of Trastuzumab, Cetuximab, Rituximab, Nivolumab, Pembrolizumab, Cemiplimab, Atezolizumab, Durvalumab, Avelumab, Ipilimumab, Relatlimab, LY3321387, MBF453, TSR-022, Urelumab, PFZ-05082566, 1-7F9 (IPH2101), GSK2831781, MED116489, MED116383, MOXR0916, Varlilumab, TRX518, NKG2D ligand-antitumour Fv fusion (preclinical development), Galunisertib, ALT-803 (IL-15-IL-15alpha-Sushi-Fc fusion complex) epacadostat, IMP321, and JNJ-83723283.

12. The method or any one of the preceding claims or the inhibitor for use of any one of the preceding claims, wherein the anti-hormonal therapy comprises an anti-estrogen therapy and/or anti-progesterone therapy.

13. The method of any one of the preceding claims or the inhibitor for use of any one of the preceding claims, wherein the tumor is a cancer, preferably a carcinoma and is most preferably selected from urothelial carcinoma, ovarian carcinoma and lung carcinoma.

14. A method for preparing a kit for predicting whether a subject having a tumor responds to a tumor treatment selected from
(i) an immunotherapy,
(ii) a chemotherapy,
(iii) an anti-hormonal therapy, and
(iv) an anti-tyrosin kinase therapy
wherein the method comprises combining
means for the detection of the level(s) of at least one nucleic acid molecule as defined in claim 1 or 2 and/or at least one protein or peptide as defined in claim 1 or 2, and instructions how to use the kit.

15. The method of claim 14, wherein the means comprise primer pairs and optionally a hydrolysis probe used for the specific detection of at least one nucleic acid molecule as defined in claim 1.


SEQUENCE LISTING (79 sequences)

SEQ ID NO: 1 - protein, 338 aa, Homo sapiens
  HLA-G membrane-bound
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 2 - protein, 341 aa, Homo sapiens
  HLA-L membrane-bound
MGVMAPRTLLLLLLGALALTETWAATPVRGWSGGRRGWSRRGWSIGTRRHGTPRATRRFTETCGPCSAITTRARPVTVRLRWQGLHRPERGPALLDRREHSGSDLPAQVGSGQILRAGQGLPEGKCMEWLRRHLENGKETLQHADPPKAHVTQHPISDHEATLRCWALGLYPAEITLTWQQDGEDQTQDTELVETRPAGDGTFQKWVAVVVPSGEEQRYMCHVQHEGLPEPLTLRWEPSSQPTIPIVGIVAGLFLLGAVVTGAVVAAAMWRKKSSGSNCAQYSDASHDTCKEDYACSCSGVCVLISFSPGCPSSLTAAGVIFPVINPTRWKAAPAHRSLWY

SEQ ID NO: 3 - protein, 319 aa, Homo sapiens
  HLA-G soluble
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 4 - protein, 246 aa, Homo sapiens
  HLA-L soluble
MGVMAPRTLLLLLLGALALTETWAATPVRGWSGGRRGWSRRGWSIGTRRHGTPRATRRFTETCGPCSAITTRARPVTVRLRWQGLHRPERGPALLDRREHSGSDLPAQVGSGQILRAGQGLPEGKCMEWLRRHLENGKETLQHADPPKAHVTQHPISDHEATLRCWALGLYPAEITLTWQQDGEDQTQDTELVETRPAGDGTFQKWVAVVVPSGEEQRYMCHVQHEGLPEPLTLRWEPSSQPTIPI

SEQ ID NO: 5 - protein, 219 aa, Homo sapiens
  HLA-J soluble
MAPRTLLLLLSGTLALAETWAGSHSMRYFSTAVSWPGRGEPSFIAVGYVDDTQFVRVDSDAVSLRMKTRARWVEQEGPEYWDLQTLGAKAQAQTDRVNLRTLLRYYNQSEADPPQDTRDPPPSLNMRHNEVLGSGLLPCGDHIDLAAGWGGPDPGHGARGDQAHRGWNLPEVGGCGSAFWRGTEIHMPCAAQGAAQAPHPEMGHTFLETSRGSKTRRFL

SEQ ID NO: 6 - protein, 221 aa, Homo sapiens
  HLA-H soluble
MVLMAPRTLLLLLSGALALTLTQTWARSHSMRYFYTTMSRPGRGEPRFISVGYVDDTQFVRFDSDDASPREEPRAPWMEREGPEYWDRNTQICKAQAQTERENLRIALRYYNQSEGGSHTMQVMYGCDVGPDGRFLRGYEQHAYDGKDYIALNEDLRSWTAADMAAQITKRKWEAARRAEQLRAYLEGEFVEWLRRYLENGKETLQRADPPQDTYDPPPHL

SEQ ID NO: 7 - DNA, 1578 bp, Homo sapiens
  HLA-G membrane-bound
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 8 - DNA, 4622 bp, Homo sapiens
  HLA-L membrane-bound
acgatcccggcactacagtcccggcgcaaccacccgcactcagattctccccaaacgccaaggatgggggtcatggctccccgaaccctcctcctgctgctcttgggggccctggccctgaccgagacctgggccgcgactccgtgagtccgaggatggagcggcgggcgccgtgggtggagcaggaggggctggagtattgggaccaggagacacggaacgccaagggccacgcgcagatttaccgagtgaacctgcggaccctgctccgctattacaaccagagcgaggccggtatgaacagttcgcctacgatggcaaggattacatcgccctgaacgaggacctgcactcctggaccgccgcgaacacagcggctcagatctcccagcacaagtgggaagcggacaaatactcagagcaggtcagggcctacctgagggcaagtgcatggagtggctccgcagacacctggagaacgggaaggagacgctgcagcacgcggatcccccaaaggcacatgtgacccagcaccccatctctgaccatgaggccaccctgaggtgctgggccctgggcctctaccctgcggagatcacactgacctggcagcaggatggggaggaccagacccaggacacggagcttgtggagaccaggcctgcaggggacggaaccttccagaagtgggtggctgtagtggtgccttccggagaggagcagagatacatgtgccatgtgcagcatgaggggctgccagagcccctcaccctgagatgggagccgtcttctcagcccaccatccccatcgtgggcatcgttgctggcctgtttctccttggagctgtggtcactggagctgtggttgctgctgcgatgtggaggaagaaaagctcaggcagcaattgtgctcagtactctgatgcatctcatgatacttgtaaagaggactatgcctgttcctgttctggtgtctgcgttctgatctctttctcccctgggtgtccctcatctctgacagcagcaggagtcatttttcctgtcattaaccccacaaggtggaaggcagcccctgcacacagaagtctgtggtattaagagatgaattttcaagcccgtgcagcttttaccctatttccagggctctttcttggattgtattttctatcttttccccaacctttttaaaggaactagattctgaaattagcagagaagagggatgccacaagttctcatcttaggtaactttctagtggaactcctcttctgctcagctctcctacccactctcccttccctgagttgtagtaatcctagcactggctctaatgcaaactcatggatctataaagcaaagtctaacttagatttatatttgtttggaaattgggattcatagtcaaagattgttctttcctaagagggaaatataattgcatgctgcagtgtgcagagggttggtgtgaaggagggatgcagggaggaagggagggaggacacacaagcagcactgctgggaaaagcacaggcggcctggatgtcagtgtgaggggaccttgtgctgtcgttgctgcaaaaccgcatttggcctgaggctatgttaataaagatactgcctttagaataggaggtgctctacagtgatgattcattcagccgacatttgctgtctgccagacatatgacagaatgtttttgcatctggggaaagtcattgaagtaaaatcagaaaaatctctagccttgtggagcatgtgttccagtgggaagaggcagacggtacatacactctaatatatgcagagtaaatgaggaaagtgttagaaggtgataagtgctgtggaacaggtgatcagagtatgggttgtgggacagagaaggtagctattgtgccggggttgtcagcgtgggccttgttgggaaggtgacctttgatgaaatatttgaaggacataaaggaatttgtcatgagggtatctggaagaagttttttctagggagtaggaaccttcagtgtcagtgtaccagggcaggatcatgtctgtgtgttctgggaagaacacgggatcgggtatggctagagcagagagtcactgagataaggtcaggggtttggtcagatcatgtgggcatagggctcaagtatgtgggaaggattttgattttgaatgagatagttttaagcagaataaagacatgccacaacttctcttttaaaaggatcactgtagctgctctgctgagaacagaatccaaaggccggcgatgagcaaggcaggtgggaaaactgtaggaaatgagtgcagtatttcaggctggagatgtcggttacttcaactggggtgtgagcagtggaaatagtgggacgtgattggattcctactatttccaatcactttataccgcattttctaatggactaaatctggggtatgagaaagaagagtaaaggataccaaaaatgtcagactgtgactaaaaagagttgccatcagctgagaatgagaagactagcaggagcatatgagaggaggggacgtcgcaggcagtcactatgggagacgtgggatctgagatgccgctgagaaataccagtgaggtagtcgggttggcagttggacagatgaatctggagacatttaggagaaatagacttgggaggtgatgtcatataacagttatttaaagccttgagtctgaatgacgtctccaagggagtgattggctgtagaagagaacaggaacaaggactgaacactaggcctctgttgctaaaggatctgatcagacaacacacctagatcagactgcacagtcctgaccccacatctagaaggtacatagaccagggagttctagactttcctgtggacaggaatcacctggacatcaccttaagtctaagctgatctggaatcgagaatgagatttcctacttatataatgttgctgttggcgctgatgctgctggtcttcagatcccacttttggtagcaagaacacagaccaggattcctaggctatgcatcagcctcgcctgtgaggcttgttaataagcaattcctgcactccatgcgcaacattctgacacaggggcatctgtggagaggcctgagtattctacaacaagcccacagcaaacctggtgctcagccagatttgatatcactgagatcagtagttggagaatgcccaggatggggaggggtctcagacccacatttaagtgttgctttattctgggttttttatttatttatttatttatttttaaggaggatgtgtttctttaattataagacaggatgctgagagataaatgtcattttctctatcatggggtatagccagatggaagattgagaagtggctcacagctcagcagaatgaaaaaatatctgaatgctgctttctgaaactactctccagaatgatttcacactcactccttggagcaaacaatgacttgcaaatttttctaatttaaacataaaggagtgtacatattggtattagtattcattttattttggggaagggcactgtattagtccatagtccgttttcacactgccgataaagacatacccaacattgggaagaaaaagaggtttaattggacttacagttccatttggctggggaggcctcagaatcatggtgggaggcgaaaggcacttcttacatggtggtggcaagagaaaatgaggaagaagcaaatgccaaaacccctgataaacacattggatctcaggagacttattcattatcatgagaatagcatgggaaagactggcccccatgattcaattacctccccctgggtccctcccacaacatgtgggaattctgggagatacaattcaagttgagatttgggtggggacacagccaaaccacattggacacagaaccaggtttgaagctacacagccaggaacataatccacagccaccctaattcagatctctcataggaaccactgtccctgctcctgagcacagatgctactgcatatacctctgataccctgatggccgacactgggccctgtggcaaagactgctatcactgctgctcctgagaactgctccactactgctcctcagccatctttaccaaaatgcagtatttactgtcccagcctctctgtgtcatctcatcctgattagaagcccacatgtggttatctaaattgtgcagccaaagcctcttgcagtgtttaactgcaataatgttggggaaagtgaatttttctcctttgtagaaggaggtagtccctgccttctaataagactcttcaacataggaagagaattcagttgctggaggtagaggggtgagggatggaaaaagaatgacaaatttcaattcctagaatcatgttctgagactagaactttatctagtacattgcaggcacctgggtttggttgagtgtataataaatgacatagttcaacttattcccttgacagtttgttttggggtccagcttttgtctaccccagttttcacacacagatacgtggagaagcattgtgtgatggtaaaatgtttacttgaaagcctttttccctatctttgtctcttgctaggattaaaaacccgtatctgt

SEQ ID NO: 9 - DNA, 1188 bp, Homo sapiens
  HLA-G soluble
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctc

SEQ ID NO: 10 - DNA, 898 bp, Homo sapiens
  HLA-L soluble
acgatcccggcactacagtcccggcgcaaccacccgcactcagattctccccaaacgccaaggatgggggtcatggctccccgaaccctcctcctgctgctcttgggggccctggccctgaccgagacctgggccgcgactccgtgagtccgaggatggagcggcgggcgccgtgggtggagcaggaggggctggagtattgggaccaggagacacggaacgccaagggccacgcgcagatttaccgagtgaacctgcggaccctgctccgctattacaaccagagcgaggccggtatgaacagttcgcctacgatggcaaggattacatcgccctgaacgaggacctgcactcctggaccgccgcgaacacagcggctcagatctcccagcacaagtgggaagcggacaaatactcagagcaggtcagggcctacctgagggcaagtgcatggagtggctccgcagacacctggagaacgggaaggagacgctgcagcacgcggatcccccaaaggcacatgtgacccagcaccccatctctgaccatgaggccaccctgaggtgctgggccctgggcctctaccctgcggagatcacactgacctggcagcaggatggggaggaccagacccaggacacggagcttgtggagaccaggcctgcaggggacggaaccttccagaagtgggtggctgtagtggtgccttccggagaggagcagagatacatgtgccatgtgcagcatgaggggctgccagagcccctcaccctgagatgggagccgtcttctcagcccaccatccccatcgtgggcatcgttgctggcctgtttctccttggagctgtggtcactggagctgtggttgctgctgcgatgtggaggaagaaaagctcag

SEQ ID NO: 11 - DNA, 1552 bp, Homo sapiens
  HLA-J soluble
ctatactatctcatgcacccaggcacaacttttccagatttaaagaaaaagaaaaaagaaataaaagaaaaaaacctctgtctctacacctccattcccagggagagctccctctctggcaccaagctccctggggtgagttttctttttgaagagtccaggggaacagcctgcgacgggtccttcttcctggacactcacgacgcggacccagttctcactcccactgagtgtcgggttttagggaagccaatcagcgtcgcgcggccccggttctaaagtccccacgcacccaccgggactcggagtctccccagacgccgacgatggggtcatggcgccccgaaccctcctcctgctgctctcggggaccctggccctggccgagacctgggcgggctcccactccatgaggtatttcagcaccgccgtttcctggccgggccgcggggagcccagcttcattgccgtgggctacgtggacgacacgcagttcgtgcgggtcgacagtgacgccgtgagtctgaggatgaagacgcgggcgcggtgggtggagcaggaggggccggagtattgggacctacagacactgggcgccaaggcccaggcacagactgaccgagtgaacctgcggaccctgctccgctactacaaccagagcgaggcggaccccccccaagacacacgtgacccacccccctctctgaacatgaggcataacgaggtcctgggttctgggcttctaccctgcggagatcacattgacctggcagcgggatggggaggaccagacccaggacatggagctcgtggagaccaggcccacaggggatggaaccttccagaagtgggcggttgtggtagtgccttctggagaggaacagagatacacatgccatgtgcagcacaaggggctgcccaagcccctcatcctgagatgggtcacacatttctggaaacttctcaaggttccaagactaggaggttcctctaggacctcatggccctgctaccttcctggcctctcacaggacgttttcttcccgcagatagaaaaggagggagctactctcaggctgcaagcagccaaagtgcccagggctctgatgtgtctctcacggcttgtaaagtgtgagacagctgccttgtgtgggactgagaggcaagatttgttcatgccttccctttgtgacttcaagaaccctgacttctctttctgcaaaggcatctgaatgtgtctgtgtccctataggcataatgtgaggtggtggggagaccagcccacacccgtgtccaccatgaccctgttccccacactgacctacattccttccccgatcacctttcctgttccagagaagtggtgctgggatgtctccatctctgtctcaacttcatggtgcactgagctgtaacttcttacttccctattaaaattagaatctgagtataaatttacttttttcaaattatttccatgacgggttgatgggttaattaaaggagaagattcctaaaatttgagagacaaaataaatggaagacatgagaa

SEQ ID NO: 12 - DNA, 1704 bp, Homo sapiens
  HLA-H soluble
agtttctcttcttctcacaacctgcgacgggtccttcttccttgatactcacgaagcggacacagttctcattcccactaggtgtcgggtttctagagaagccaatcggtgccgccgcggtcccggttctaaagtccccacgcacccaccgggactcagattctccccagacgccgaggatggtgctcatggcgccccgaaccctcctcctgctgctctcaggggccctggccctgaccctgacccagacctgggcgcgctcccactccatgaggtatttctacaccaccatgtcccggcccggccgcggggagccccgcttcatctccgtcggctacgtggacgatacgcagttcgtgcggttcgacagcgacgacgcgagtccgagagaggagccgcgggcgccgtggatggagcgggaggggccagagtattgggaccggaacacacagatctgcaaggcccaggcacagactgaacgagagaacctgcggatcgcgctccgctactacaaccagagcgagggcggttctcacaccatgcaggtgatgtatggctgcgacgtggggcccgacgggcgcttcctccgcgggtatgaacagcacgcctacgacggcaaggattacatcgccctgaacgaggacctgcgctcctggaccgcggcggacatggcagctcagatcaccaagcgcaagtgggaggcggcccgtcgggcggagcagctgagagcctacctggagggcgagttcgtggagtggctccgcagatacctggagaacgggaaggagacgctgcagcgcgcggaccccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccgagcccctcaccctgagatgggagccatcttcccagcccaacgtccccatcgtgggcatcgttgctggcctggttctacttgtagctgtggtcactggagctgtggtcgctgctgtaatgtggaggaagaagagctcagatagaaaaggagggagctactctcaggctgcaacagcaacagtgcccagggctctgatgtgtctctcacggcttgaaagtgtgagacagctgccttgtgtgggactgagaggcaagagttgttcctgccttccctttgtgacttgaagaaccctgactttctttctacaaaggcacctgaatgtgtctgtgttcctgtaggcataatgtgtggaggaggggagaccaacccaccctcatgtccaccatgaccctcttccccacgctgatctgtgttccctccccaatcatctttcctgttccagagaggaggggctgagatgtctccatctttttctcaactttatgtgcactgagctgtaacttcttacttccctcttaaaattagaatctgagtaaacatttactttttcaaattcttgccatgagaggttgatgacttaattaaaggagaagattcctaaaatttgagagacaaaataaatggaacacatgagaaccttccag

SEQ ID NO: 13 - protein, 4 aa, Artificial Sequence
  redox-active site
CGPC

SEQ ID NO: 14 - DNA, 19 bp, Artificial Sequence
  Forward Primer HLA-G Ex3
ggccggagtattgggaaga

SEQ ID NO: 15 - DNA, 23 bp, Artificial Sequence
  Probe HLA-G Ex3
caaggcccacgcacagactgaca

SEQ ID NO: 16 - DNA, 20 bp, Artificial Sequence
  Reverse Primer HLA-G Ex3
gcagggtctgcaggttcatt

SEQ ID NO: 17 - DNA, 19 bp, Artificial Sequence
  Forward Primer HLA-G Ex4
ctgcggctcagatctccaa

SEQ ID NO: 18 - DNA, 21 bp, Artificial Sequence
  Probe HLA-G Ex4
cgcaagtgtgaggcggccaat

SEQ ID NO: 19 - DNA, 23 bp, Artificial Sequence
  Reverse Primer HLA-G Ex4
caggtaggctctcctttgttcag

SEQ ID NO: 20 - DNA, 24 bp, Artificial Sequence
  Forward Primer HLA-G Ex5
caccaccctgtctttgactatgag

SEQ ID NO: 21 - DNA, 21 bp, Artificial Sequence
  Probe HLA-G Ex5
accctgaggtgctgggccctg

SEQ ID NO: 22 - DNA, 24 bp, Artificial Sequence
  Reverse Primer HLA-G Ex5
agtatgatctccgcagggtagaag

SEQ ID NO: 23 - DNA, 20 bp, Artificial Sequence
  Forward Primer HLA-G Ex6
catccccatcatgggtatcg

SEQ ID NO: 24 - DNA, 22 bp, Artificial Sequence
  Probe HLA-G Ex 6
tgctggcctggttgtccttgca

SEQ ID NO: 25 - DNA, 20 bp, Artificial Sequence
  Reverse Primer HLA-G Ex6
ccgcagctccagtgactaca

SEQ ID NO: 26 - DNA, 22 bp, Artificial Sequence
  Forward Primer HLA-G Ex8
gaccctcttcctcatgctgaac

SEQ ID NO: 27 - DNA, 28 bp, Artificial Sequence
  Probe HLA-G Ex8
cattccttccccaatcacctttcctgtt

SEQ ID NO: 28 - DNA, 19 bp, Artificial Sequence
  Reverse HLA-G Ex8
catcccagccccttttctg

SEQ ID NO: 29 - DNA, 19 bp, Artificial Sequence
  Forward Primer HLA-G Ex3-5
ttcatcgccatgggctacg

SEQ ID NO: 30 - DNA, 22 bp, Artificial Sequence
  Probe HLA-G Ex3-5
cgacacgcagttcgtgcggttc

SEQ ID NO: 31 - DNA, 19 bp, Artificial Sequence
  Reverse Primer HLA-G Ex3-5
atcctcggacacgccgagt

SEQ ID NO: 32 - DNA, 20 bp, Artificial Sequence
  Forward Primer HLA- G Ex2/3
ccgaaccctcttcctgctgc

SEQ ID NO: 33 - DNA, 20 bp, Artificial Sequence
  Probe HLA-G Ex2/3
cgagacctgggcgggctccc

SEQ ID NO: 34 - DNA, 20 bp, Artificial Sequence
  Reverse Primer HLA-G Ex2/3
gcgctgaaatacctcatgga

SEQ ID NO: 35 - DNA, 19 bp, Artificial Sequence
  Forward Primer HLA-H Ex 2/3
gagagaacctgcggatcgc

SEQ ID NO: 36 - DNA, 24 bp, Artificial Sequence
  Probe HLA-H Ex2/3
agcgagggcggttctcacaccatg

SEQ ID NO: 37 - DNA, 19 bp, Artificial Sequence
  Reverse Primer HLA-H Ex2/3
ccacgtcgcagccatacat

SEQ ID NO: 38 - DNA, 19 bp, Artificial Sequence
  Forward Primer HLA-H
gagagaacctgcggatcgc

SEQ ID NO: 39 - DNA, 25 bp, Artificial Sequence
  Probe HLA-H
accagagcgagggcggttctcacac

SEQ ID NO: 40 - DNA, 16 bp, Artificial Sequence
  Reverse Primer HLA-H
cgggccgggacatggt

SEQ ID NO: 41 - DNA, 18 bp, Artificial Sequence
  Forward Primer KRT 5
cgccacttaccgcaagct

SEQ ID NO: 42 - DNA, 24 bp, Artificial Sequence
  Probe KRT5
tggagggcgaggaatgcagactca

SEQ ID NO: 43 - DNA, 25 bp, Artificial Sequence
  Reverse Primer KRT5
acagagatgttgactggtccaactc

SEQ ID NO: 44 - DNA, 26 bp, Artificial Sequence
  Forward Primer KRT20
gcgactacagtgcatattacagacaa

SEQ ID NO: 45 - DNA, 32 bp, Artificial Sequence
  Probe KRT20
ttgaagagctgcgaagtcagattaaggatgct

SEQ ID NO: 46 - DNA, 21 bp, Artificial Sequence
  Reverse Primer KRT20
cacaccgagcattttgcagtt

SEQ ID NO: 47 - DNA, 17 bp, Artificial Sequence
  Forward Primer PD-1
ggccagcccctgaagga

SEQ ID NO: 48 - DNA, 23 bp, Artificial Sequence
  Probe PD-1
acccctcagccgtgcctgtgttc

SEQ ID NO: 49 - DNA, 22 bp, Artificial Sequence
  Reverse Primer PD-1
ggaaatccagctccccatagtc

SEQ ID NO: 50 - DNA, 23 bp, Artificial Sequence
  Forward Primer PDL1
tggcatccaagatacaaactcaa

SEQ ID NO: 51 - DNA, 30 bp, Artificial Sequence
  Probe PDL1
caaagtgatacacatttggaggagacgtaa

SEQ ID NO: 52 - DNA, 24 bp, Artificial Sequence
  Reverse Primer PDL1
ttgaagatcagaagttccaatgct

SEQ ID NO: 53 - DNA, 24 bp, Artificial Sequence
  Forward Primer FGFR1
aattcaaacctgaccacagaattg

SEQ ID NO: 54 - DNA, 25 bp, Artificial Sequence
  Probe FGFR1
aggctacaaggtccgttatgccacc

SEQ ID NO: 55 - DNA, 24 bp, Artificial Sequence
  Reverse Primer FGFR1
caccacagagtccattatgatgct

SEQ ID NO: 56 - DNA, 19 bp, Artificial Sequence
  Forward Primer FGFR2
aagcaggagcatcgcattg

SEQ ID NO: 57 - DNA, 28 bp, Artificial Sequence
  Probe FGFR2
aggctacaaggtacgaaaccagcactgg

SEQ ID NO: 58 - DNA, 21 bp, Artificial Sequence
  Reverse Primer FGFR2
cagatgggaccacactttcca

SEQ ID NO: 59 - DNA, 19 bp, Artificial Sequence
  Forward Primer FGFR3
agcgcgtactgtgccactt

SEQ ID NO: 60 - DNA, 23 bp, Artificial Sequence
  Probe FGFR3
agtgtgcgggtgacagacgctcc

SEQ ID NO: 61 - DNA, 20 bp, Artificial Sequence
  Reverse Primer FGFR3
ctccccgtcttcgtcatctc

SEQ ID NO: 62 - DNA, 20 bp, Artificial Sequence
  Forward Primer FGFR4
ggatggacaggcctttcatg

SEQ ID NO: 63 - DNA, 21 bp, Artificial Sequence
  Probe FGFR4
cattggaggcattcggctgcg

SEQ ID NO: 64 - DNA, 21 bp, Artificial Sequence
  Reverse Primer FGFR4
cacgagactccagtgctgatg

SEQ ID NO: 65 - DNA, 20 bp, Artificial Sequence
  Forward Primer CALM2
gagcgagctgagtggttgtg

SEQ ID NO: 66 - DNA, 22 bp, Artificial Sequence
  Probe CALM2
tcgcgtctcggaaaccggtagc

SEQ ID NO: 67 - DNA, 21 bp, Artificial Sequence
  Reverse Primer CALM2
agtcagttggtcagccatgct

SEQ ID NO: 68 - DNA, 21 bp, Artificial Sequence
  Forward Primer HLA-L Ex2/3
cctgctccgctattacaacca

SEQ ID NO: 69 - DNA, 26 bp, Artificial Sequence
  Probe HLA-L Ex2/3
cgaggccggtatgaacagttcgccta

SEQ ID NO: 70 - DNA, 20 bp, Artificial Sequence
  Reverse Primer HLA-L Ex2/3
cgttcagggcgatgtaatcc

SEQ ID NO: 71 - DNA, 18 bp, Artificial Sequence
  Forward Primer HLA-L Ex5/6
gctgtggttgctgctgcg

SEQ ID NO: 72 - DNA, 29 bp, Artificial Sequence
  Probe HLA-L Ex5/6
agaaaagctcaggcagcaattgtgctcag

SEQ ID NO: 73 - DNA, 30 bp, Artificial Sequence
  Reverse Primer HLA-L Ex5/6
catagtcctctttacaagtatcatgagatg

SEQ ID NO: 74 - DNA, 23 bp, Artificial Sequence
  Forward Primer HLA-L Ex7
tcctcttctgctcagctctccta

SEQ ID NO: 75 - DNA, 35 bp, Artificial Sequence
  Probe HLA-L Ex7
ctctcccttccctgagttgtagtaatcctagcact

SEQ ID NO: 76 - DNA, 27 bp, Artificial Sequence
  Reverse Primer HLA-L Ex7
gctttatagatccatgagtttgcatta

SEQ ID NO: 77 - DNA, 17 bp, Artificial Sequence
  Forward Primer HLA-J Ex4/5
caaggggctgcccaagc

SEQ ID NO: 78 - DNA, 30 bp, Artificial Sequence
  Probe HLA-J Ex4/5
catcctgagatgggtcacacatttctggaa

SEQ ID NO: 79 - DNA, 27 bp, Artificial Sequence
  Reverse Primer HLA-J Ex4/5
cctcctagtcttggaaccttgagaagt